(19) United States
(12) Patent Application Publication
(10) Pub. No.: US 2011/0177619 A1
(43) Pub. Date: Jul. 21, 2011

(54) IN VITRO DIAGNOSTIC MARKERS COMPRISING CARBON NANOPARTICLES AND KITS

(76) Inventors: Andrew Metters, Clemson, SC (US); Qian Wang, Columbia, SC (US); Siqi Li, Clemson, SC (US); Hui Hu, Woburn, MA (US)

(21) Appl. No.: 13/003,843

(22) PCT Filed: Jul. 14, 2009
(86) PCT No.: PCT/US09/04060

§ 371 Date: Apr. 4, 2011

Related U.S. Application Data
(60) Provisional application No. 61/080,479, filed on Jul. 14, 2008.

(51) Int. Cl. G01N 33/543; C07D 495/04; C07D 475/04; G01N 21/76; C40B 40/06; G01J 1/00; B82Y 15/00

(52) U.S. Cl. 436/518; 548/256; 544/261; 436/172; 506/16; 356/213; 977/773; 977/920

(57) ABSTRACT

This invention relates to luminescent markers for in vitro diagnostic applications, and kits using those markers. In some embodiments, those markers comprise luminescent carbon nanoparticles. Some embodiments provide a method for investigating an analyte comprising correlating a marker to the analyte and observing the luminescence from the marker, wherein the marker comprises a nanoparticle having a carbon core. In vitro kits, including those employing a marker comprising a nanoparticle having a carbon core, are also provided.

### RELATED APPLICATIONS

This application claims benefit of priority under PCT Chapter I, Article 8, and 35 U.S.C. §119(e) of U.S. Provisional Application No. 61/080,479, entitled “IN VITRO DIAGNOSTIC MARKERS COMPRISING CARBON NANOPARTICLES AND KITS,” filed on Jul. 14, 2008, which is incorporated herein by reference in its entirety.

### TECHNICAL FIELD

This invention relates to luminescent markers for in vitro diagnostic applications, and kits using those markers. In some embodiments, the markers comprise luminescent carbon nanoparticles.

### BACKGROUND ART

Many diagnostic technologies currently use radioactive labels, fluorescent organic dyes, and fluorescent semiconductor quantum dots. For example, biologically-active compounds labeled with radioactive isotopes are routinely employed to image diseased tissue, both inside (in vivo) and outside (in vitro) patients. Fluorescent organic dyes covalently bound to biologically-active compounds also provide medically-useful imaging means. Recently, fluorescent semiconductor quantum dots, or nanocrystals, have been discovered as useful labels as well. Typically, those quantum dots contain a core of cadmium selenide, indium phosphide, indium arsenide, lead sulfide, lead selenide, or other semiconductor, often capped with a less-toxic material. The less-toxic material often has an energetic band gap larger than the core semiconductor to avoid interfering with the fluorescence of the core.

U.S. Pat. No. 7,235,361 to Bawendi et al. discloses the use of fluorescent semiconductor nanocrystals, also called quantum dots, in various applications to label biological targets. The semiconductor materials disclosed in the '361 patent include binary, tertiary, and quaternary semiconductors from groups II, III, IV, and V of the Periodic Table, as well as Ge and Si. The '361 patent also describes protocols for employing fluorescent quantum dots in place of radiolabeling and organic fluorescent dyes for many biological and medical applications, and those protocols are incorporated herein by reference. The '361 patent does not describe luminescent carbon nanoparticles.

U.S. Pat. No. 6,468,808 and U.S. Pat. No. 7,192,785, both to Nie et al. describe water-soluble quantum dots for biological applications. Those quantum dots are made soluble by providing a hydrophilic attachment group with the quantum dot. Neither the '808 patent nor the '785 patent describe luminescent carbon nanoparticles.

U.S. Patent Application Publication No. 2007/0082411 to Muys describes methods and an apparatus for detecting bioconjugates of fluorescent quantum dots. The methods involve separating quantum dots conjugated to a biological material from nonconjugated quantum dots using a filter. The fluorescence from the conjugated quantum dots reveals information about the biological material. Those quantum dots are described as “inorganic semiconductor nanocrystals.” Luminescent carbon nanoparticles do not appear in the '411 publication.

Known labeling technologies, such as radioactive labels, fluorescent organic dyes, and fluorescent semiconductor quantum dots, have some shortcomings. Safety, toxicity, and the potential to pollute the environment temper their use. Moreover, strict excitation parameters, photobleaching, and weak fluorescence signals render some of those labeling technologies difficult to use. Accordingly, new luminescent markers are needed for diagnostic applications.

### DISCLOSURE OF THE INVENTION

The present invention relates, in some aspects, to the use of luminescent carbon nanoparticles such as those described in PCT Application No. PCT/US06/42233 for in vitro diagnostic uses. The '233 application, which published on May 3, 2007 as PCT publication no. WO2007/050984, is incorporated herein by reference.

Some embodiments of the present invention provide a method of investigating at least one analyte in vitro, comprising: correlating the at least one analyte with at least one marker, and observing the luminescent emission of the at least one marker. In further embodiments, the marker comprises at least one binding agent. In still further embodiments, the marker comprises at least one chromophore. In additional embodiments, the at least one marker comprises at least one nanoparticle comprising at least one carbon core and at least one passivation agent coupled to the at least one carbon core, wherein the at least one carbon core is less than about 100 nm in size.

In further embodiments, the luminescent emission of the at least one marker increases in the presence of the at least one analyte. In those embodiments, the analyte “de-quenches” or enhances the luminescence of the marker.

In still further embodiments, the luminescent emission of the at least one marker decreases in the presence of the at least one analyte. In those embodiments, the analyte quenches the luminescence of the marker.

Additional embodiments provide a method of investigating at least one analyte in vitro, comprising: correlating the at least one analyte with at least one marker; and observing the luminescent emission of the at least one marker; wherein the at least one marker comprises at least one nanoparticle comprising at least one carbon core and at least one passivation agent coupled to the at least one carbon core, wherein the at least one carbon core is less than about 100 nm in size, and wherein the luminescent emission is chosen from chemiluminescence, electroluminescence, thermal luminescence, sonoluminescence, and combinations thereof.

Certain embodiments of the present invention provide a method of investigating at least one analyte in vitro, comprising: correlating the at least one analyte with at least one marker, wherein the correlating comprises forming at least one sandwich complex; and observing the luminescent emission of the at least one marker; wherein the at least one marker comprises at least one nanoparticle comprising at least one carbon core and at least one passivation agent coupled to the at least one carbon core, and wherein the at least one carbon core is less than about 100 nm in size. Thus, in some embodiments, the sandwich complex comprises at least one immobilized antibody; the at least one analyte, which comprises at least one antigen, bound to the at least one immobilized antibody; and the at least one marker, which comprises at least one additional antibody, bound to the at least one antigen. In further embodiments, the sandwich complex comprises at least one immobilized antigen; the at least one analyte, which comprises at least one primary antibody, bound to the at least one immobilized antigen; and the at least one marker, which comprises at least one secondary antibody, bound to the at least one primary antibody.

Yet other embodiments provide a method of investigating at least one analyte in vitro, comprising: correlating the at least one analyte with at least one marker; and observing the correlation of the at least one analyte with the at least one marker with at least one interaction chosen from magnetic interaction, electrical interaction, light absorption, light scattering, and combinations thereof; wherein the at least one marker comprises at least one nanoparticle comprising at least one carbon core and at least one passivation agent coupled to the at least one carbon core, and wherein the at least one carbon core is less than about 100 nm in size.

Still further embodiments provide a marker for in vitro diagnosis, comprising: at least one carrier particle; at least one biologically active agent coupled to the carrier particle and adapted to correlate with at least one analyte; and at least one nanoparticle coupled to the carrier particle and comprising at least one carbon core and at least one passivation agent coupled to the at least one carbon core, wherein the at least one carbon core is less than about 100 nm in size, and wherein the at least one nanoparticle is luminescent.

Some embodiments discovered during the investigations of the present invention relate to markers comprising at least one binding agent and at least one chromophore. Further embodiments relate to markers comprising at least one binding agent, at least one chromophore, and at least one nanoparticle comprising at least one carbon core and at least one passivation agent coupled to the at least one carbon core, wherein the at least one carbon core is less than about 100 nm in size, and wherein the at least one nanoparticle is luminescent.

Some embodiments provide a kit for in vitro diagnosis, comprising: at least one marker that is adaptable to correlate with at least one analyte. In certain embodiments the at least one marker comprises at least one binding agent and at least one chromophore. In further embodiments, the at least one marker comprises at least one nanoparticle comprising at least one carbon core and at least one passivation agent coupled to the at least one carbon core, wherein the at least one carbon core is less than about 100 nm in size, and wherein the nanoparticle is luminescent.

Some embodiments provide a kit for in vitro diagnosis, comprising: at least one marker that is adaptable to correlate with at least one analyte; wherein the at least one marker comprises at least one nanoparticle comprising at least one carbon core and at least one passivation agent coupled to the at least one carbon core, wherein the at least one carbon core is less than about 100 nm in size; and

wherein the at least one marker exhibits increased luminescence either in the presence or absence of the analyte.

### BRIEF DESCRIPTION OF THE DRAWINGS

FIG. 1 shows a TEM image of nanoparticles comprising PEG passivation agent at approximately 200,000× magnification.

FIG. 2 shows steady state photoluminescence spectra of nanoparticles comprising PEG passivation agent taken as the excitation wavelength varied by 20 nm increments from 360 to 600 nm.

FIG. 3 shows the relative cell viability for five human cell lines in the presence of varying concentrations of nanoparticles after 24 hours' incubation, according to the MTT assay.

FIG. 4 shows a color-inverted composite image of the photoluminescence under channel pass filters (DAPI, FITC and Cy3) from KB cells incubated 24 hours with markers comprising a carbon core with a PEG passivation agent, a folate binding agent and a hydrophilic fluorescein chromophore.

### MODES FOR CARRYING OUT THE INVENTION

Reference will now be made in detail to various embodiments of the disclosed subject matter, one or more examples of which are set forth below. Each embodiment is provided by way of explanation, not limitation, of the subject matter. In fact, it will be apparent to those skilled in the art that various modifications and variations may be made without departing from the scope or spirit of the disclosure. For instance, features illustrated or described as part of one embodiment may be used in another embodiment to yield a still further embodiment.

As used herein, in vitro diagnostics include any use outside of a living being other than single-celled organisms for detecting, monitoring, identifying, analyzing, isolating, diagnosing, measuring, or otherwise investigating one or more analytes. Analytes subject to the present invention can come from any source, such as, for example, biological, environmental, industrial, and even extraterrestrial sources. Analytes include, but are not limited to, organisms and portions and products thereof; bacteria, viruses, prions, and portions and products thereof; DNA and fragments thereof; RNA and fragments thereof; proteins and fragments thereof; drugs, nutrients, poisons, toxins, and metabolites thereof; chemicals; minerals; pollutants; explosives, propellants, accelerants, and combusted residues thereof; trace elements and trace compounds; substances useful in forensic investigations; and raw materials, reactants, products, and impurities from industrial processes. In some embodiments of the present invention, analytes include any matter that can correlate, or can be adapted to correlate, with a marker.

An analyte may correlate with a marker of the present invention in any manner. Correlation, in some embodiments of the present invention, means the accumulation of analyte and marker together. Correlation can involve, but is not limited to, chemical bonding, such as covalent bonding and ionic bonding; van der Waals interaction; dipole-dipole interaction; static charge attraction; and magnetic attraction.

Some embodiments of the present invention provide correlation between an analyte and a marker to a degree merely discernibly greater than the correlation between non-analytes and the marker. In other embodiments, there is a high degree of correlation between the analyte and the marker. In yet other embodiments, there is substantially no correlation between non-analytes and the marker. In still other embodiments, a given marker will exhibit different degrees of correlation to different analytes. Non-analytes include, for example, any matter from which the investigator wishes to distinguish an analyte. Some embodiments include more than one analyte. Some embodiments include more than one marker.

Correlation also can involve, in some embodiments of the present invention, a marker in a quenched state (i.e., non-luminescent) being converted to a non-quenched state (i.e., luminescent) in the presence of an analyte. In such embodiments, the accumulation of a marker with an analyte need not be greater than the accumulation of the marker with non-analytes. In still other embodiments, the analyte quenches the marker, and the degree of quenching reveals the presence or concentration of the analyte. A marker is luminescent in certain embodiments if the marker exhibits any detectible luminescence.

The markers of some embodiments of the present invention comprise at least one carbon core. The at least one carbon core can comprise any form of carbon. For example, the carbon core can include amorphous carbon, crystalline carbon, fullerene carbon, other nanocarbon, or a combination thereof. The carbon cores of the nanoparticles of the present invention also can be any suitable size. In some embodiments the carbon core has a size (i.e., diameter) less than about 100 nm. The carbon core can be smaller, in some embodiments. For example, the carbon core can be less than about 30 nm in size, or between about 1 nm and about 10 nm in size. The markers of the present invention, include, for example, the fluorescent carbon nanoparticles disclosed in PCT application no. PCT/US06/42233.

Coupled to the carbon core can be a passivation agent. A passivation agent can be, for example, a molecule, a polymer or a biopolymer. The passivation agent can be coupled to the carbon core in any suitable fashion such as, for example, covalent bonding between the two, non-covalent bonding, and combinations thereof. In some embodiments, a passivation agent can retain a reactive functionality. For example, after coupling to a carbon core, a passivation agent can retain an amino group useful to attach further moieties such as chromophores, binding agents, and linkers.

A luminescent nanoparticle as described herein can include additional materials. For example, a material (e.g., a metal or a magnetic material) can be embedded in or on the carbon core. In some embodiments, a member of a specific binding pair can be bound to the passivation agent, for instance via a reactive functional chemistry retained on the passivation agent following binding of the passivation agent to the carbon core. The member of the specific binding pair bound to the passivation agent is known as a binding agent.

Methods for making markers according to the present invention can include, for instance, forming a carbon core, for example via laser ablation of graphite or electric arc discharge of a carbon powder. A formation method can include coupling a passivation agent to a carbon core according to any suitable method. In some embodiments, a formation method can include binding an additional material, for instance a member of a specific binding pair, to a carbon nanoparticle, for instance via the passivation agent.

A core carbon nanoparticle can be formed according to any suitable process capable of forming a carbon particle on a nanometer scale. For example, in some embodiments, a core carbon nanoparticle can be formed from an amorphous carbon source, such as carbon black; from graphite, for instance in the form of graphite powder; from nanocarbon, such as fullerenes, nanotubes, nanorods, nano-onions, and nanohorns; or from crystalline carbon (e.g., diamond). For example, according to some embodiments, a core carbon nanoparticle can be formed according to a laser ablation method from a graphite starting material. In other embodiments, a core carbon nanoparticle can be formed in an electric arc discharge from carbon powders. Other methods can be utilized as well, for instance, thermal carbonization of particles of carbon-rich polymers. Such methods are generally known to those of ordinary skill in the art and thus are not described in detail herein.

A carbon nanoparticle can generally be any size from about 1 nm to about 100 nm in average diameter. While not wishing to be bound by any particular theory, it appears that there is quantum confinement effect on the observed luminescence of the materials, and in particular, a relatively large surface area to volume ratio may be helpful to confine the recombination of excitons to the surface of a nanoparticle. Accordingly, it appears that higher luminescence quantum yields can be achieved with a smaller core carbon nanoparticle as compared to a larger nanoparticle having the same or similar surface passivation. As such, a luminescent particle including a relatively larger core carbon nanoparticle, e.g., greater than about 30 nm in average diameter, can be less luminescent than a smaller particle. In some embodiments, a core carbon nanoparticle can be less than about 20 nm in average diameter, for instance, in some further embodiments, between about 1 and about 10 nm in average diameter.

In some embodiments, a carbon core can include other components, in addition to carbon. For example, metals and/or other elements can be embedded in a carbon core. In other embodiments, a magnetic metal alone or in combination with other materials, such as, for example, Ni/Y, can be embedded in a carbon core. For example, the addition of the desired materials, e.g., a metal powder, to the carbon core can be attained through the addition of the materials during the formation process of the carbon particles and the material can thus be incorporated into the core. Upon the functionalization of such a nanoparticle to provide surface passivation, the resulting luminescent carbon nanoparticle that includes an embedded metal, e.g., an embedded magnetic metal, can be magnetically responsive.

A passivation agent can be any material that can bind to a carbon nanoparticle surface and encourage or stabilize the radiative recombination of excitons, which is believed to come about through stabilization of the excitation energy ‘traps’ existing at the surface as a result of quantum confinement effects and the large surface area to volume ratio of a nanoparticle. One or more passivation agents can be bound to a nanoparticle surface according to any binding methodology. For example, a passivation agent can bind to a nanoparticle surface covalently or noncovalently or a combination of covalently and noncovalently. Moreover, a passivation agent can be polymeric, molecular, biomolecular, or any other material that can passivate a nanoparticle surface. For instance, the passivation agent can be a synthetic polymer such as poly(lactic acid) (PLA), poly(ethylene glycol) (PEG), polyoxyalkyleneamine, poly(propionylethylenimine-co-ethylenimine) (PPEI-EI), and poly(vinyl alcohol) (PVA). In some embodiments, the passivation agent can be a biopolymer, for instance a protein or peptide. Other exemplary passivation agents can include molecules bearing amino and other functional groups. Certain embodiments provide monoamino passivation agents, while other embodiments provide diamino passivation agents. Passivation agents can be any suitable molecular weight, and a given carbon core can have more than one passivation agent, and passivation agents that are alike or different having different molecular weights.

The passivation agent and/or additional materials grafted to the core nanoparticle via the passivation agent can provide the luminescent particles with additional desirable characteristics. For example, a hydrophilic passivation agent can be bound to the core carbon nanoparticle to improve the solubility/dispersibility of the nanoparticles in water. In other embodiments, a passivation agent can be selected so as to improve the solubility of the carbon nanoparticle in an organic solvent. In still other embodiments, a passivation agent can be selected to improve the solubility of the carbon nanoparticle in water or other polar solvent.

Markers of the present invention can correlate to analytes according to any suitable method. In some embodiments, the passivation agent of the marker is adapted to bind to the analyte. For example, the passivation agent may contain a binding agent that is adapted to covalently or ionically bond with one or more binding sites on the analyte. In another example, the passivation agent contains moieties such as hydroxyl groups that hydrogen-bond with the analyte. In yet another example, the marker contains a magnetic structure that is adapted to magnetically bind with the analyte, which also contains a magnetic structure. In some embodiments, the analyte is immobilized, and free marker is introduced, correlates with the immobile analyte, and any remaining free marker is washed away. Correlated marker, now immobilized with the analyte, luminesces under excitation, thereby revealing the analyte. In other embodiments, the marker is immobilized, and free analyte is allowed to correlate with the immobile marker.

Markers of the present invention can be induced to luminesce through any suitable method. In some embodiments, the marker is made to achieve an energetically excited state, and then the marker achieves a lower energy state by releasing some or all of the energy stored in the excited state. When some or all of the released energy takes the form of light energy, the marker is said to luminesce. The excited state can be achieved, for example, by applying one or more forms of energy to the marker, such as, for example, light, electrical, chemical, thermal, vibrational, mechanical, and magnetic energy. In some embodiments, light of sufficient energy causes a marker to achieve an excited state, and the marker then photoluminesces. In further embodiments, more than one photon is absorbed, leading to multiphoton photoluminescence. In other embodiments, the analyte is a reactive species capable of transferring energy to the marker, thereby causing the marker to chemiluminesce. In still other embodiments, an electric field causes the marker to electroluminesce. Yet other embodiments provide thermoluminescent markers, while still other embodiments provide sonoluminescent markers.

Some markers of the present invention comprise at least one nanoparticle, and at least one species chosen from antigens, antibodies, hormones, DNA fragments, polysaccharides, proteins, peptides, cell-surface receptors, fractions of any of the foregoing, or a combination of two or more of any of the foregoing, bound to the at least one nanoparticle. Those species can be alike or different on a given nanoparticle; in some embodiments, there are more than one such species. Those species can function as the passivation agent, or the nanoparticle can include one or more passivation agents distinct from those species. Thus, a nanoparticle of the present invention can provide a scaffold for numerous species that are alike or different. Those species can function as binding agents in certain embodiments. Among binding agents, biotin, folic acid, and streptavidin, and derivatives thereof may be mentioned. In further embodiments, binding agents are chosen from Protein A, immunoglobulin-binding proteins, haptens particular for a given antibody, complete antigens, and epitopes of antigens, and combinations thereof.

In additional embodiments, a nanoparticle of the present invention comprises one or more chromophores, such as, for example, organic dyes including, but not limited to fluorescein, rhodamine, and coumarin dyes; for example fluorescein, fluorescein isothiocyanate (“FITC”), coumarin, rhodamine, pyrene, and anthracene; semiconductor quantum dots including, but not limited to, cadmium selenide, indium phosphide, indium arsenide, lead sulfide, lead selenide; and the like. Those embodiments of the present invention, among others, provide markers having considerable versatility for detecting and therefore investigating analytes. Making such markers can follow any suitable procedure, including those described herein for making other markers of the present invention.

As can be appreciated, more than one binding agent, and/or more than one chromophore, can be employed in markers of the present invention.

Luminescence from markers according to the present invention can be observed by any suitable method. In some embodiments, a photodetector sensitive to a narrow band of light corresponding to the emission expected from a marker correlated with a given analyte is placed near a sample under excitation. The photodetector is calibrated so that any signal above the noise from the photodetector indicates the presence of analyte, in that example. In a similar embodiment, the photodetector is calibrated to indicate the concentration of analyte. In other embodiments, one or more photodetectors are arranged to record the emission spectrum of the marker. In still other embodiments, a sample contains more than one marker, more than one analyte, or a combination thereof, which yield a complex emission spectrum that is recorded and analyzed to reveal information about the sample. Some embodiments provide a diffraction grating to spectrally analyze the emission. Some embodiments provide one or more signal analyzers to resolve the emission. Still other embodiments provide a means such as, for example, an array of photodetectors to image the emission from a sample. Still other embodiments provide a means to scan a sample, and further means to assemble an image from the scan.

In some embodiments of the present invention, as mentioned elsewhere, the presence of the analyte increases or decreases the luminescence of the marker. In other words, in certain embodiments, the marker correlating with the analyte causes the marker to luminesce with greater or lesser intensity. In some embodiments, the analyte completely quenches the marker's luminescence. In other embodiments, the analyte allows a completely quenched marker to luminesce.

Some embodiments of the present invention provide a marker comprising a carbon nanoparticle and a quencher coupled to the carbon nanoparticle, for example, through a covalent linker. The quencher can be any species that accepts energy from the carbon nanoparticle, thereby stopping or diminishing the luminescent emission from the carbon nanoparticle. The marker is designed so that, when correlated with an analyte, energy transfer between the nanoparticle and the quencher is stopped or diminished, thereby allowing the marker to luminesce and reveal the presence of the analyte. In some embodiments, the quencher is chosen from molecular species such as, for example, N,N-diethylaniline and nitrobenzene. In other embodiments, two carbon nanoparticles are coupled together, so that one nanoparticle quenches the other in the absence of the analyte. In some embodiments, the presence of the analyte changes the physical conformation of the nanoparticle relative to the quencher, thereby affecting the quencher's effect on luminescence. In other embodiments, the presence of the analyte severs the coupling between the nanoparticle and the quencher, thereby affecting the quencher's effect on luminescence. In some of those embodiments, the linker contains at least one labile moiety easily severable such as, for example, an ether linkage, an ester linkage, an amide linkage, or a dithiol linkage. Further embodiments provide linkers that modify the hydrophilicity of the marker. In certain cases, for example, an ether linkage, an ester linkage, an amide linkage, or a dithiol linkage may modify the hydrophilicity of a marker. Other examples of suitable linkers include but are not limited to alkyne linker, azide linker, 1,11-diazido-3,6,9-trioxaundecane, and 1-amino-11-azido-3,6,9-trioxaundecane and combinations thereof. It will be appreciated, for example from the chemistry described below, that a named linker might not retain its original structure once it has performed linking chemistry. As shown in the Examples, an alkyne linker does not retain the alkyne structure once a linkage is formed, yet the resulting marker is still said to contain an alkyne linker.

In still other embodiments, the presence of the analyte changes the emission spectrum of the marker. In those embodiments, the marker correlated with the analyte emits light of different wavelength(s) compared to the uncorrelated marker. In some embodiments, the emission spectrum shifts to higher or to lower energy upon correlation, and the analyte is investigated based on the shifted emission spectrum. In other embodiments, the analyte changes the nature of the emission transition, such as, for example, by converting a singlet-singlet transition (fluorescence) into a triplet-singlet transition (phosphorescence). In certain of those embodiments, the analyte is investigated based on the lifetime of the decay of the transition.

Further embodiments of the present invention provide a method for investigating at least one analyte in vitro comprising correlating the at least one analyte with at least one marker, and detecting that correlation using other than luminescence. For example, the light absorbance or light scattering exhibited by a marker can indicate the correlation. In another example, a nanoparticle of the present invention comprising a magnetic material can indicate the presence, distribution, or concentration of a correlated analyte using MRI imaging, or other magnetic interaction with the marker. A further example provides the electrochemical oxidation or reduction of a nanoparticle in a marker correlated with an analyte, and that oxidation or reduction is detected by current, potential, optical absorbance, or other phenomenon that does not include luminescence. In some of those examples, the marker can comprise at least one nanoparticle comprising at least one carbon core and at least one passivation agent coupled to the at least one carbon core, wherein the at least one carbon core is less than about 100 nm in size.

Some embodiments of the present invention provide a kit comprising at least one marker for at least one analyte. Such kits may have any degree of sophistication, ranging from simple kits that can be purchased over the counter and used by a consumer at home, to more complicated kits to be used by persons with advance training such as laboratory technicians. In some embodiments, a kit provides at least one marker adapted to correlate with at least one analyte.

For example, a kit provides at least one marker immobilized on a structure such as a plate, pad, stick, slide, or other device, and the user of the kit would apply a substance containing at least one analyte to the structure. Suitable substances for these and other embodiments include bodily fluids, solids, and tissues, for example, including urine, saliva, blood, stool, mucous, semen, menstrual fluids, body cavity rinsings, tissue scrapings, and tissue biopsies, among others. By applying the substance to the structure, the user allows the at least one analyte to correlate with the at least one marker. Then the user in that example would send the structure with the at least one marker to a designated place such as a laboratory so the structure can be analyzed. Optionally, the kit in that example can be adapted to allow the user to analyze the structure himself, by providing an excitation source such as, for example, a black light, electrical device, sonicator, or chemical agent to induce luminescence from the correlated marker. Or, in some embodiments, the analyte correlating to the marker induces chemiluminesce. In some embodiments, the structure comprises a chemiluminescent agent, while in other embodiments, a chemiluminescent agent is provided separately from the structure to be added after correlation between the marker and the analyte.

### EMBODIMENTS

Immunoassays (Various Formats)

In one embodiment, the principle components of an immunoassay can be covalently or non-covalently labeled with a luminescent marker such as a luminescent carbon-core nanoparticle (e.g., Selah Dots® available at www.SelahTechnologies.com) to indicate the presence and/or quantity of biomolecular recognition events specific to the assay of interest, for example, through an optical or photoluminescent signal. The principle components of an immunoassay include one or more of immobilized antigens, free antigens, primary antibodies, secondary antibodies, enzymes, and other intermediate compounds. Suitable reactive functionalities and methods for binding or conjugating the luminescent carbon nanoparticles to the principle assay components are generally known to those of skill in the art. For example, reactive functionalities on the passivation agent of the luminescent carbon nanoparticle can be used to tag or label target molecules through amine or thiol moieties present within the structure of those molecules to form suitable markers. In another instance, carbon cores conjugated to streptavidin proteins can be bound non-covalently to biotinylated macromolecules such as antigens or antibodies through the natural and selective binding of streptavidin for the small molecule biotin, to form further markers. In another instance, carbon cores modified with Protein A or other immunoglobulin-binding proteins can be used to optically label the primary or secondary antibodies used in the immunoassay. In the foregoing instances, the streptavidin proteins, Protein A, and immunoglobulin-binding proteins can act as the passivation agent, and/or the nanoparticle can include another passivation agent(s). In some embodiments, suitable ligands for labeling of antibodies include haptens particular for that antibody, complete antigens, and epitopes of antigens. In certain embodiments of the present invention, the principle components of an immunoassay labeled with a marker provide the means by which an analyte will correlate with the marker.

Luminescent carbon nanoparticle markers can be utilized for immunoassay reactions in a variety of formats.

For instance, markers of the present invention can label the antigens or antibodies in heterogeneous competitive or non-competitive immunoassays. In a heterogenous immunoassay, one component can be immobilized on a structure as described above. For instance, in a non-competitive format, the presence of a particular antigen in a sample is assessed by first immobilizing an antibody for that antigen on a structure. The antigen-containing sample is incubated with the immobilized antibody on the structure such that all of the antigen molecules bind, but not all of the antibody sites are occupied. To detect the amount of antigen attached to the antibody, a second antibody labeled with, for example, luminescent carbon nanoparticles (the marker) is added which binds to another epitope of the antigen, forming a sandwich complex. After washing off any excess reagent, the sandwich complexes containing the luminescent markers can be detected and the luminescent signal generated is directly related to the amount of antigen present in the sample. If the anticipated concentration of the antigen is greater than the available immobilized antibody, the sample can be diluted in some embodiments.

Luminescent carbon nanoparticle markers can be used with lateral flow immunoassays to detect the presence of specific antigens in various bodily fluids such as blood or urine. In one instance, pregnancy can be detected by the presence of the glycoprotein hormone human chorionic gonadotropin (hCG) in the urine. In one example, this type of assay is carried out on a test strip and based on the sandwich format with two antibodies. One antibody, the capture antibody, is immobilized to the test strip. A second antibody, the tracer antibody, is labeled with one or more luminescent carbon nanoparticles to form a marker. The tracer antibody is impregnated into the surface of the structure but is not permanently attached. When a liquid sample potentially containing the antigen of interest is applied to the test strip containing both antibodies, the biomolecular recognition reactions are carried out in the flow. If the antigen of interest is present in the sample it will form a complex with the labeled tracer antibody. This complex continues to move along the test strip and passes over the immobilized capture antibody. A sandwich complex is formed between the immobilized capture antibody, the antigen in the sample, and the labeled tracer antibody, thereby correlating the analyte with the marker and immobilizing both for observation of luminescence. The amount of sandwich complexes formed is directly proportional to the amount of antigen present in the sample. The complexes labeled with the luminescent carbon nanoparticles can be detected via the absorbance or scattering of ambient or incident light. The labeled complexes can also be detected by irradiating photoluminescent carbon nanoparticles with UV, visible, near-IR, or IR light to generate a photoluminescent signal proportional to the number of immobilized antigen molecules.

In another instance, the presence and concentration of a specific antibody in a sample can be detected via immunoassay. For example, HIV antibodies are only produced when an infection with the virus occurs. Antigens that bind specifically to the antibodies of interest are immobilized on a structure, in a further embodiment of the present invention. The sample, possibly containing the antibodies of interest, is incubated with the antigen-presenting structure. After a washing step to remove any unbound materials, a secondary antibody labeled with the luminescent carbon nanoparticles (the marker) is added. The secondary antibody binds to the primary sample antibody, usually to the Fc region of the primary antibody, thereby correlating the analyte with the marker. After removing any unbound secondary antibody, the luminescence from the marker can be detected and directly correlated to the amount of primary antibody in the original sample.

Fret (Including Use in Immunoassays, as Molecular Beacons, and in Real-Time PCR)

In other embodiments, the luminescence of carbon nanoparticles can be quenched or enhanced in the presence of a particular targeted substance to indicate the presence or absence of a particular analyte, the occurrence or absence of a particular binding event, or a change in molecular conformation under a variety of environmental conditions. For example, this behavior can be utilized in an assay format to detect biomolecular recognition events via Fluorescence Resonance Energy Transfer (FRET) between two molecules or epitopes that demonstrate affinity or otherwise interact with one another. In one instance, a luminescent carbon nanoparticle is used to label one of the two molecules or molecule fragments involved in the recognition event, to form a marker. This molecule or fragment could be an antigen, an antibody, a hormone, a DNA fragment, a polysaccharide, protein, peptide, cell-surface receptor, or other molecule or fragment. A substance capable of quenching the optical signal produced by the photoluminescent carbon nanoparticle is attached to the other, unlabeled molecule or molecule fragment involved in the recognition event, which will function as the analyte. Quenching of the luminescent carbon nanoparticle signal is used to indicate correlation of the analyte with the marker, and therefore the binding or localization of the two molecules or molecule fragments of the binding pair. This method of binding detection can be incorporated into high throughput screening assays used to rapidly identify, for example, lead compounds with specific biological activity from large libraries of small molecules, natural product extracts, proteins, and peptides, in additional embodiments of the present invention.

In another instance, a luminescent carbon nanoparticle and quenching species can be attached to the same molecule. For example, the proximity of the quencher reduces the luminescence emitted by the carbon nanoparticle under certain conditions due to FRET. In another instance, the luminescent carbon nanoparticle and quencher are attached to complimentary arm ends of a so-called molecular beacon, a single-stranded oligonucleotide hybridization probe that forms a stem-and-loop structure, to form a marker. Molecular beacons comprising luminescent markers can be utilized as optical probes for use in diagnostic assays designed for genetic screening, SNP detection, and pharmacogenetic applications. The loop contains a probe sequence that is complementary to a target oligonucleotide sequence (the analyte), and the stem is formed by the annealing of complementary arm sequences that are located on either side of the probe sequence. Molecular beacons comprising luminescent markers do not fluoresce to any significant extent when they are free in solution. However, when they hybridize to a nucleic acid strand containing a target sequence, they undergo a conformational change that increases the distance between the luminescent marker and quencher, enabling the luminescent carbon nanoparticles to luminesce brightly. In some embodiments, molecular beacons comprising luminescent markers can be used as amplicon probes for the diagnostic assay of complimentary DNA strands during polymerase chain reaction (PCR). Because nonhybridized molecular beacons are dark, it is not necessary to isolate the probe-target hybrids (i.e., correlated analyte-markers) to determine the number of amplicons synthesized during an assay. Molecular beacons are added to the assay mixture before carrying out gene amplification and luminescence intensity can be measured in real time in a closed, homogeneous system, in certain embodiments.

Molecular beacons comprising luminescent carbon nanoparticles that luminesce at different wavelengths enable assays to be carried out that simultaneously detect different targets in the same reaction. For example, multiplex assays can contain a number of different oligonucleotide primer sets, each set enabling the amplification of a unique gene sequence from a different pathogenic agent. A corresponding number of molecular beacons can be present as markers, each containing a probe sequence specific for one of the amplicons, and each labeled with a luminescent carbon nanoparticle of a different color of luminescence. The color of the resulting luminescence, if any, identifies the pathogenic agent in the sample. In additional embodiments, the number of amplification cycles required to generate detectable fluorescence provides a quantitative measure of the number of target organisms present. If more than one type of pathogen is present in the sample, the luminescence colors that occur identify which are present. Luminescence colors, and emission spectra in general, can be modified by adjusting the passivation agent chemistry, size of the carbon core, or a combination thereof.

In another instance of the present invention, the luminescence of a luminescent marker is minimized or eliminated by the quencher during hybridization of a short, tagged oligonucleotide to a target DNA sequence. During primer extension, the luminescent marker is cleaved from the probe molecule due to the exonuclease activity of the polymerase. No longer in the proximity of the quenching agent, the luminescent marker luminesces in the reaction mixture under appropriate excitation. The intensity of the signal is directly proportional to the number of amplified DNA molecules.

DNA Binding Arrays

In another embodiment, luminescent markers can be used to indicate hybridization of complimentary DNA strands present in a DNA binding array. In a DNA binding array, large numbers of single-stranded DNA molecules or oligonucleotides are immobilized onto a structure such as a glass slide or nylon membrane in the form of microscopic spots. In one instance, a DNA binding array is treated with a sample solution containing single stranded DNA fragments that have been labeled with luminescent carbon nanoparticles. If the labeled, sample DNA fragments (markers) are complimentary to any sequence present in the array (analytes), the sample DNA hybridizes to the immobilized DNA fragment, thereby correlating analytes with markers. If no complimentary sequence is found, the sample DNA stays in solution and is washed away in the next reaction step. The result of this procedure is that non-hybridized spots on the array remain colorless while the hybridized ones will luminesce according to the properties of the attached luminescent carbon nanoparticles. Using luminescent markers such as carbon nanoparticles with DNA binding arrays, it is possible to identify the sequence of a gene and discover gene mutations or so-called single nucleotide polymorphisms (SNPs) that may be important for identifying disease or assessing risk factors associated with a disease. In addition to their utility in DNA sequencing, DNA binding arrays based on luminescent carbon nanoparticle probes may also provide useful applications in diagnostics, pharmacogenomics, expression profiling, and toxicology. For example, the DNA of normal cells can be compared to diseased cells or cells treated with drugs. The binding signature of genomic DNA from different cells can also be compared for gene discovery and polymorphism analysis. In another instance, RNA binding arrays monitored via luminescent carbon nanoparticle labels can be used for protein expression profiling.

Cell and Tissue Imaging (Including Flow Cytometry)

In further embodiments, luminescent markers can be utilized to tag or mark the presence of a particular substance, ligand or receptor in a cell or tissue sample. In one instance, the presence, quantity and location of specific analytes in histologically prepared tissue samples can be identified using luminescent carbon nanoparticles labeling appropriate antigen or antibody fragments or whole molecules. In another instance, live cells can be imaged using modified or unmodified luminescent carbon nanoparticles. For example, incubating live cells in a solution of PEGylated carbon nanoparticle markers leads to fluid-phase uptake of the particles via passive diffusion, endocytosis, and/or other mechanisms. Cytosolic distribution of the luminescent carbon nanoparticle markers within the cell permits imaging of the entire cell via fluorescence microscopy or flow cytometry. In another instance, luminescent carbon nanoparticles can be modified with biologically active or therapeutic molecules that enable their binding or localization to specific sub-cellular compartments such as the nucleus. This localized binding permits localized imaging of specific sub-cellular compartments or intracellular tracking of the labeled molecule.

In a further instance, markers of the present invention can be used in tumor margin assessment. For example, a surgeon may excise what is believed to be the entire tumor block from a patient. The entire surface of the tumor block can contact a composition comprising markers of the present invention which are adapted to bind to the cancer cells of the tumor. Then the tumor block can be observed for luminesce from the bound markers, perhaps with imaging technology to record the observation. If the tumor block shows a cohesive fringe of healthy tissue surrounding the tumor block, the surgeon can conclude that the entire tumor has been removed. If, however, the tumor block lacks a fringe of healthy tissue, the surgeon may conclude that some diseased tissue remains in the patient. In certain of these embodiments, the entire surface of the tumor block can be assessed. In the current state of the art, only 10-15% of the surface is assessed for a fringe of healthy tissue.

In another instance, luminescent carbon nanoparticles can be modified with molecules that target specific cell-membrane receptor molecules or ligands. These modified luminescent carbon nanoparticles can then be used to identify and quantify specific cell types or cells expressing certain receptor molecules or ligands. In a further instance, the luminescence intensity provided by the carbon nanoparticles bound to specific cells can be detected with a flow cytometry and cell sorting device to quantify and/or sort cells based on their type, age, or disease status. In one additional instance, the luminescent signal obtained from the tagged cells can be amplified by first doping polymeric nanoparticles or microparticles with the luminescent carbon nanoparticles. The carbon-doped polymeric particles can then be functionalized with the biologically active agents and used to label the cell-surface receptor molecules or ligands in a similar fashion as described above. Accordingly, the present invention provides markers for in vitro diagnosis, comprising at least one carrier particle, at least one biologically active agent coupled to the carrier particle and adapted to correlate with at least one analyte, and at least one nanoparticle coupled to the carrier particle and comprising at least one carbon core and at least one passivation agent coupled to the at least one carbon core, wherein the at least one carbon core is less than about 100 nm in size. In certain embodiments, the at least one nanoparticle is luminescent. A biologically active agent is a binding agent that allows the marker to act as a biomarker, labeling an analyte of biological or medical significance.

### EXAMPLES

### Example 1

### Preparation of Carbon Core

Carbon particles were produced by laser ablation of graphite powder carbon in the presence of water vapor, in accordance with the methods set forth in Y. Suda et al., Thin Solid Films, 415, 15 (2002), which is entirely incorporated by reference herein. The carbon particles were refluxed in 2.6 M aqueous nitric acid for 12 hours. The nitric acid reflux provides carboxylic acid groups on the surface of the carbon. The carbon cores range in size from 2 to 7 nm as seen by TEM (not shown).

### Example 2

### Coupling Passivation Agent to Carbon Core

One of three passivation agents were chosen:

(a) O,O′-bis(3-aminopropyl)poly(ethylene glycol), H₂NCH₂CH₂CH₂(CH₂CH₂O)ₙCH₂CH₂CH₂NH₂ (n=31) (PEG, MW=1500);
(b) polyoxyalkyleneamine (ED, Jeffamine ED900, MW=900); or
(c) poly(propionylethylenimine-co-ethylenimine) (PPEI-EI, MW=50,000, EI mole fraction 15%).
In further experiments, a fourth passivation agent was used: poly(propionylethylenimine) (PPEI).
The carbon cores were mixed with the chosen passivation agent in a 1 to 10 ratio by mass with DMF as solvent and agitated at 120° C. for 3-6 days, cooled, diluted with water, and centrifuged. To facilitate covalent bonding between the passivation agent and the nanoparticle, the carbon core with carboxylic acid groups was first reacted with thionyl chloride to convert to acylated carbon, which led to covalent amidation when reacted with the chosen passivation agent having amine groups. To facilitate non-covalent bonding between the passivation agent and the nanoparticle, the carbon core with carboxylic acid groups was reacted with the passivation agent containing amine groups without further modification. The supernatant was collected, containing the nanoparticles. Scheme I depicts some possible coupling mechanisms of the passivation agents to the carbon core to form the nanoparticles:

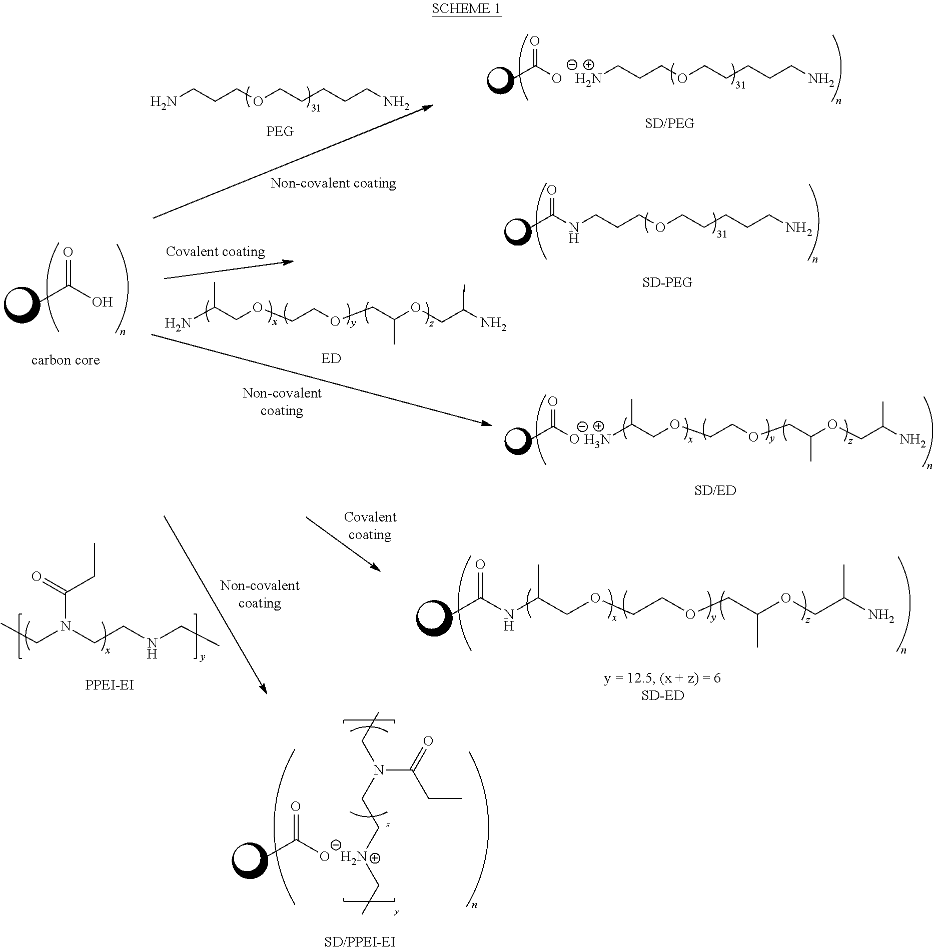

In Scheme I, the variables n, x, y, and z are any suitable numbers. In some cases, n is determined by the number of carboxylic acid sites on the surface of a carbon core which is controlled by the oxidation conditions and particle size distribution. For the passivation agent labeled ED used in the present examples, y is on average 12.5, while x+z=6. For the passivation agent labeled PPEI-EI, x ranged from 400 to 450 and y ranged from 50 to 100. These parameters were varied by controlling the reaction conditions, primarily reaction time.

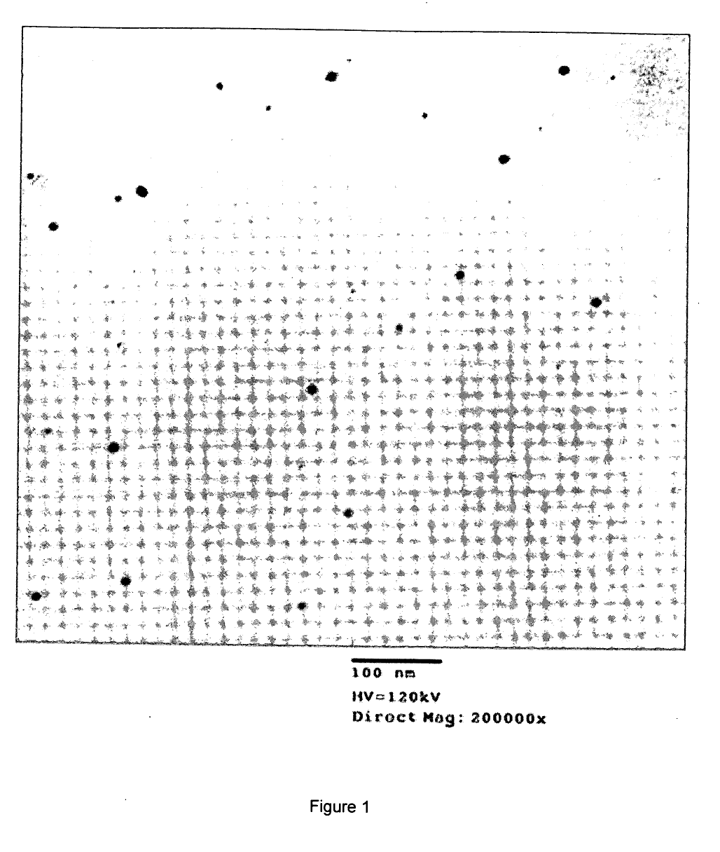

Nanoparticles comprising PEG or ED ranged in size from 5 to 26 nm as calculated from size exclusive elution volume chromatography, and from 3 to 5 nm when the nanoparticles comprise PPEI-EI. FIG. 1 shows nanoparticles comprising PEG passivation agent under 200,000× magnification. Average particle size is 9±2.5 nm.

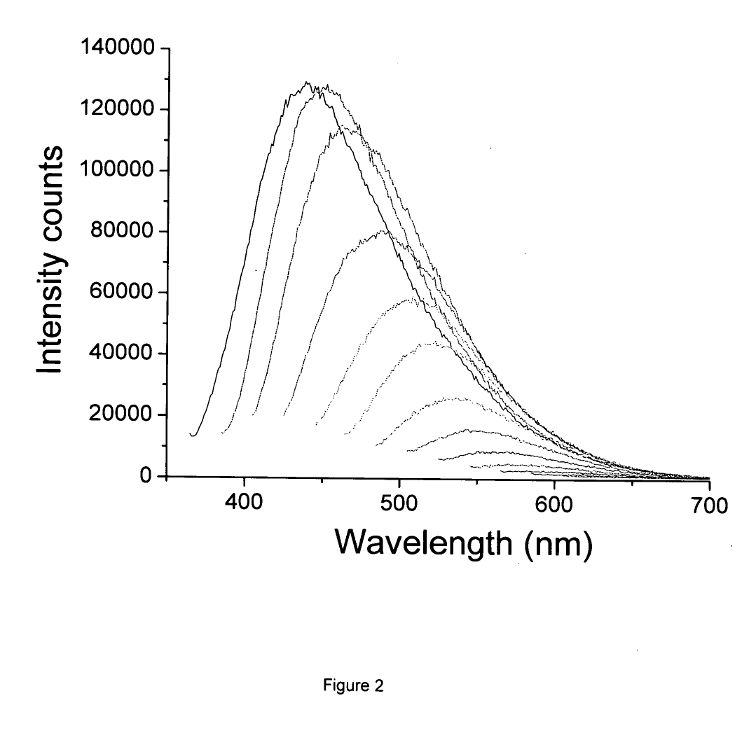

Exhibiting a broad excitation wavelength range from at least 360 nm to about 600 nm, the nanoparticles demonstrated high extinction coefficients on the order of 10⁶ M⁻¹·cm⁻¹ which compares favorably to <10⁵ M⁻¹·cm⁻¹ extinction coefficient for many organic dyes. Quantum yield ranged between 1 and 10%. Steady state fluorescence spectra of nanoparticles comprising PEG passivation agent are shown in FIG. 2. Excitation wavelength is 360-600 nm at 20 nm increments.

### Example 3

### Cytotoxicity Studies of Nanoparticles

To measure the viability of cells in the presence of nanoparticles, the CellTiter-Blue® Cell Viability Assay (Promega, WI) was chosen. In that assay, cells are incubated in the presence of resazurin, a compound having relatively low fluorescence. Viable cells convert resazurin via metabolic reduction by enzymes such as NADP and FADH to highly fluorescent resorufin. By measuring the relative intensities of fluorescence by resorufin, experiments were conducted that probe the cytotoxicity of nanoparticles having various passivation agents.

Chinese hamster ovary (“CHO”) cells, HeLa cells, NIH 3T3 fibroblast cells were grown in cell culture incubators with a 5% CO₂ atmosphere in 96-well tissue culture plates at 37° C. using Dulbecco's Modified Eagle Medium and passaged at confluence. After the cells attached to the wells, they were washed twice with 100 μL of medium, and then 100 μL suspensions of medium containing nanoparticles at given concentrations were added to the wells. Cell viabilities were assessed by adding 20 μL of CellTiter-Blue® Cell Viability Assay solutions to each well and incubation continued for a given number of hours, such as 1, 3, 6, 12, 24, 48, and 96 hours. The fluorescent emission was measured at excitation wavelength (“λex”) 560 nm and emission wavelength (“λem”) 590 nm. Human lines MDA-MB3, MDA-MB4, HUVA, HASM, and HeLa were grown under similar conditions and tested using the MTT assay, in which (3-(4,5-dimethylthiazol-2-yl)-2,5-diphenyltetrazolium bromide (“MTT”) is metabolized by viable cells into formazan, yielding information similar to the CellTiter-Blue® assay. The positive control in the MTT assay contained no nanoparticles.

Two controls were included with these experiments simultaneously. (1) The positive control: the cells were incubated with medium and CellTiter-Blue® Cell Viability Assay only; (2) The negative control: the medium contained nanoparticles and CellTiter-Blue® Cell Viability Assay only. Separately, a third control was performed: the passivation agents were incubated with cells, medium, and CellTiter-Blue® Cell Viability Assay without nanoparticles, to assess the cytotoxicity of the passivation agents.

Since the cell viability is proportional to the fluorescence intensity, the cell viability as a percentage of the positive control was calculated from the fluorescence intensity value at λem 590 nm which was corrected for background fluorescence. The fluorescence intensity was normalized according to that of the positive control, in order to show the relative activity of the samples.

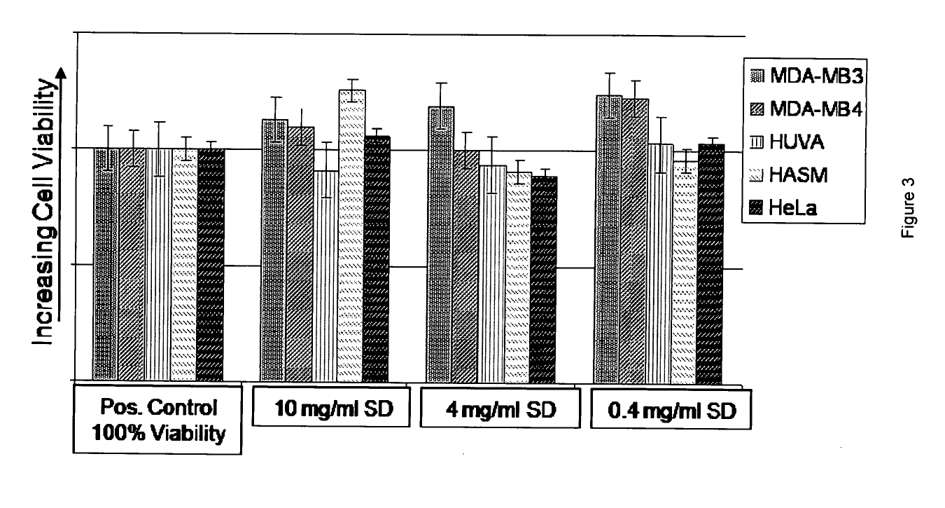

FIG. 3 shows the relative cell viability in the presence of nanoparticles comprising PEG passivation agent after 24 hours' incubation according to the MTT assay. The figure presents data for the cell lines MDA-MB3, MDA-MB4, HUVA, HASM, and HeLa. Also tested were the cell lines CHO and NIH-3T3 (not shown) and nanoparticles comprising PPEI-EI or ED passivation agents by the CellTiter-Blue® Cell Viability Assay. No concentration of nanoparticles tested extinguished cell viability. All cell lines were viable in the presence of the nanoparticles, metabolizing MTT into formazan in the MTT assay, and resazurin into resorufin in the CellTiter-Blue® Cell Viability Assay. Nanoparticles comprising PPEI-EI as a passivation agent showed a concentration-dependent reduction of fluorescence in HeLa and CHO cells, with cell viability appearing the same as the positive control below about 0.004 mg/mL. Nanoparticles comprising PEG as a passivation agent showed no effect on HeLa cell viability at 24 hours, but then an inverse concentration-dependent reduction of fluorescence appeared at 48 hours. NIH 3T3 cells showed lower fluorescence versus positive control after two hours for certain passivation agents, but then demonstrated enhanced fluorescence greater than positive control for PEG and ED. For some experiments, fluorescence intensity decreased for nanoparticle concentration above about 1 mg/mL, reaching about 50% of positive control at about 10 mg/mL concentration of nanoparticles. These results indicate that passivation agents and concentrations can be selected to minimize the appearance of cytotoxic effects, if any, while maximizing the ability to mark target cells.

The passivation agents by themselves showed no cytotoxic trends at any concentration, for any cell line, according to the CellTiter-Blue® Cell Viability Assay. Many showed an enhancement of fluorescence versus control, which is believed to be caused by an accumulation of the chromophore in the passivation agent.

### Example 4

### Attaching Binding Agent to Nanoparticle

Two binding agents, biotin and folic acid, were attached to separate nanoparticles according to the following procedures using amine-n-hydroxysuccinamide chemistry. Biotin is known to bind tenaciously to avidins, and is useful for targeting proteins, cancer cells, and polynucleotides in laboratory assays. Also useful for targeting cancer cells, folic acid and its derivatives can enter cells through the folate receptor (FR), a 38 kDa glycosylphosphatidylinositol anchored glycoprotein, through mediated endocytosis, and by non-specific endocytosis. That ability can facilitate cell labeling by nanoparticles in accordance with certain embodiments of the present invention.

### Example 4A

### Biotinylation of Nanoparticles

Biotin-NHS ester was prepared as follows:

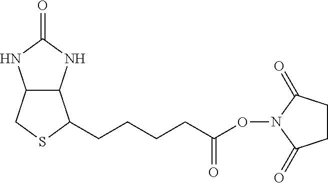

To a round bottom flask with a stirring bar, biotin (2.0 g, 8.2 mmol) was added to dimethylformamide (“DMF”) (60 mL) and heated to dissolve. N-hydroxysuccinimide (“NHS”) (0.944 g, 8.2 mmol) and N,N′-dicyclohexylcarbodiimide (“DCC”) (2.2 g, 10.7 mmol) were added to the clear solution, the flask was capped and stirred at room temperature overnight. The white liquid was filtered and DMF was evaporated to small amount. Diethyl ether (“ether,” Et₂O) was added and stirred. The white solids were recrystallized from isopropanol to give 2.7 g (97%) product. ¹H NMR (300 MHz, DMSO-d₆) δ=6.40 (1H, s, NH), 6.34 (1H, s, NH), 4.29 (1H, m, bridge CH), 4.14 (1H, m, bridge CH), 3.09 (1H, m, thiophene CH), 2.82 (1H, m, thiophene CH₂), 2.79 (4H, s, NHS CH₂), 2.67 (2H, m, CH₂C═O), 2.57 (1H, m, SCH), 1.63-1.42 (6H, m, CH₂CH₂CH₂). See Susumu, K.; Uyeda, H. T.; Medintz, I. L; Pons, T.; Delehanty, J. B; Mattoussi, H. Enhancing the stability and biological functionalities of quantum dots via compact multifunctional ligands. J. Am. Chem. Soc. 2007, 129, 13987-96, which is entirely incorporated herein by reference.

Nanoparticles were biotinylated as follows.

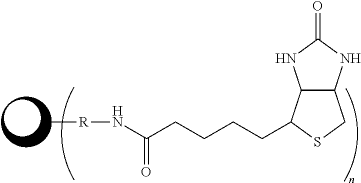

NaCl was added to nanoparticles comprising PEG passivation agent in aqueous solution until it was saturated. R in the structure above represents PEG passivation agent, and n is any suitable positive number. Dichloromethane (“DCM”) was added to extract the nanoparticles three times and dried by Na₂SO₄. DCM solution was filtered and evaporated and further dried in vacuo to give 14 mg (9.3E-6 mol based on PEGDA1500) black residue. Biotin NHS ester (43 mg, 126E-6 mol), DMF (0.5 mL) and triethylamine (“TEA”) (0.06 mL) were added and all reactants dissolved. The flask was capped and stirred at room temperature for 3 days. Saturated NaHCO₃ was added to degrade excess biotin NHS ester for 2 h. NaCl was added to the solution until it was saturated. It was extracted by DCM three times. The DCM layer was washed with saturated NaHCO₃ 3 times, water 3 times and brine once, dried by Na₂SO₄, filtered, evaporated, and dried in vacuo to remove anything volatile.

The black residue was redissolved into saturated NaHCO₃ which was saturated by NaCl and extracted by DCM three times. The DCM layer was washed with saturated NaHCO₃ aqueous solution three times, water three times and brine once, dried by Na₂SO₄, filtered, evaporated, and dried in vacuo to remove any volatile to give the final product (10 mg, 71% yield). ¹H NMR showed the absence of biotin NHS ester and the presence of biotin in the product. ¹H NMR (300 MHz, CDCl₃): δ=4.52 (1H, m, bridge CH), 4.33 (1H, m, bridge CH), 3.65 (br, CH₂O), 3.16 (1H, m, thiophene CH), 2.93 (1H, m, thiophene CH₂), 2.74 (1H, m, CHS), 2.37 (2H, m, CH₂C═O), 1.99 (br, residue NH₂), 1.79 (2H, m, CH₂CHS), 1.69 (2H, m, CH₂CH₂C═O), 1.24 (2H, m, CH₂CH₂CH₂C═O).

### Example 4B

### Folation of Nanoparticles

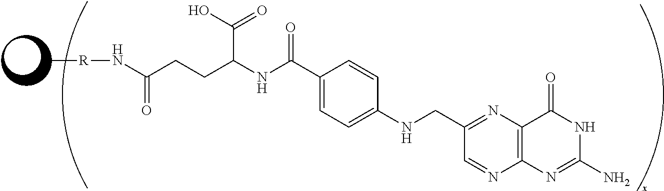

Commercially available anhydrous folic acid (9.3 mg, 21E-6 mol) was dissolved in a composition comprising PEG-coated nanoparticles in DMSO (1 mL), followed by addition of 1-ethyl-3-(3-dimethylaminopropyl) carbodiimide) (“EDC”) (5.4 mg, 28E-6 mol), hydroxybenzotriazole (“HOBt”) (4.3 mg, 28E-6 mol), and triethylamine (0.02 mL) was added after 24 hours. R in the structure above represents PEG passivation agent, and x is any suitable positive number. After 48 hours, the reaction was quenched by a suitable amount of HCl (1 M). The mixture was extracted with DCM, and the organic layer was washed by brine and dried by Na₂SO₄. After evaporation, black solids were recovered (20 mg, 75% yield). The folic acid carboxyl groups were activated by EDC and HOBt. The participation of TEA as a base to remove a proton appears to facilitate the coupling between folic acid and the amine group of PEG passivation agent. NMR confirmed the attachment of the folate to the nanoparticle, but the signal-to-noise ratio was not optimal.

### Example 4C

### Adding Streptavidin Binding Agent to Nanoparticles

Proteins such as streptavidin can act as a binding agent between a marker of the present invention and, for example, biotinylated antibodies bound to target analytes such as cancer cells or antigens.

Activating nanoparticles with BS3: A solution of nanoparticles comprising PEG passivation agent in 0.1M PBS buffer, pH 7.4, was prepared at a concentration of 2.5 mg/ml. Bis[sulfosuccinimidyl]suberate (“BS3”) (Pierce) was dissolved in 0.1M PBS buffer, pH 7.4 at a concentration of 25 mM. The solution of nanoparticles comprising PEG passivation agent and the BS3/PBS solution were mixed (25 equiv. of Nanoparticles/PEG) in a round bottom flask with a stir bar. The mixture reacted under stirring at room temperature for 0.5 hour. The BS3 activated particles was purified by gel filtration (PD 10 column) using PBS buffer to remove excess BS3.

Attaching streptavidin to nanoparticles activated with BS3 was accomplished as follows: An aqueous solution of streptavidin (“SAv”) (Cell Sciences product, MW 53 kD) with a concentration of 25 mg/ml was added to the purified BS3 activated particle PBS solution (20-25 equiv. of Nanoparticles/PEG) in a round bottom flask with a stir bar. The mixture reacted under stirring at room temperature for 2 hours. The reaction was quenched with 1M glycine under stirring for 15 minutes. The Nanoparticles/PEG/Streptavidin (SAv) was separated from the free excess SAv by size exclusion column (XK 16/30 Superose 6 column) using PBS buffer.

The number of SAv per particle was determined by Coomassie protein assay kit (Pierce), which is a quantitative method for total protein. Specifically, when coomassie dye binds protein in an acidic medium, an immediate shift in absorption maximum occurs from 465 nm to 595 nm with a concomitant color change from brown to blue. A small amount of Nanoparticles/PEG/Streptavidin (SAv) sample was mixed with the assay reagent and measured the absorbance at 595 nm. SAv concentration in the sample was estimated by reference to absorbances obtained for a series of standard SAv dilutions, which were assayed alongside the unknown samples. The number of SAv molecules per particle was varied by controlling the reactant stoichiometry.

### Example 5

### Attaching Chromophore to Nanoparticle

The following chromophores can be attached to nanoparticles comprising PEG passivation agent: fluorescein, Rhodamine B, pyrene, and anthracene. In one approach, the chromophore would be functionalized with an n-hydroxysuccinamide leaving group at the desired site of attachment according to the same reaction employed to functionalize biotin, described above, yielding a chromophore NHS ester. Then the chromophore NHS ester would be attached directly to the nanoparticle, via the amine groups present on the PEG passivation agent, according to this reaction:

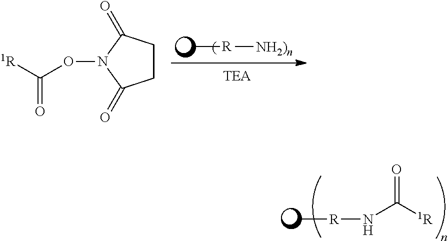

In that reaction, R represents the passivation agent, ¹R represents the chromophore NHS ester, and n is a suitable positive number. Chromophore NHS esters can be represented as follows:

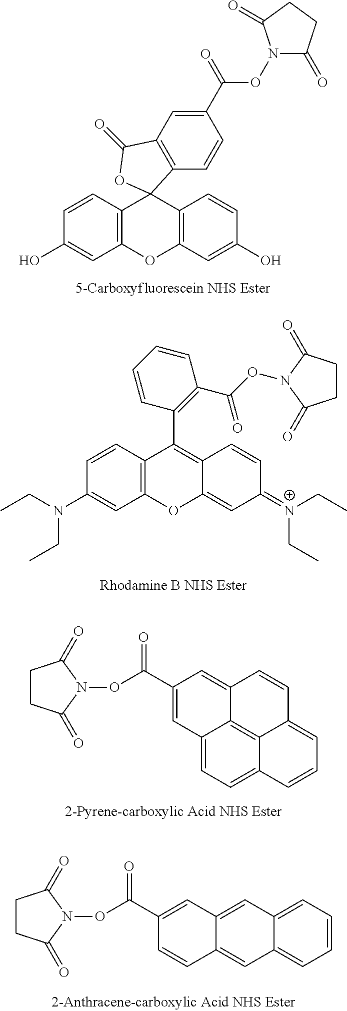

### Example 5A

### Attaching Fluorescein Directly to Nanoparticle Comprising PEG Passivation Agent

To a round bottom flask with a stirring bar, nanoparticles comprising PEG passivation agent, 5-carboxyfluorescein NHS ester (10 equiv.), DMF and triethylamine can be added, capped and stirred at room temperature for 3 days. The reaction is quenched by stirring with saturated NaHCO₃ solution for 2 hr or more. The aqueous phase is then saturated with NaCl and extracted by DCM three times. The organic phase would be washed by saturated NaHCO₃ solution three times, then water, brine, and dried by Na₂SO₄, filtered and evaporated to remove all volatiles. The residue can be dissolved in saturated NaHCO₃ aqueous solution. The aqueous phase can be saturated with NaCl and extracted by DCM three times. The organic phase can be washed by saturated NaHCO₃ solution three times, then by water, brine, and dried by Na₂SO₄, filtered and evaporated to dryness to give the final product.

### Example 5B

### Attaching Rhodamine B Directly to Nanoparticle Comprising PEG Passivation Agent

To a round bottom flask with a stirring bar, nanoparticles comprising PEG passivation agent, Rodamine B NHS ester (10 equiv.), DMF and triethylamine can be added, and the flask would be capped and stirred at room temperature for 3 days. The reaction can be quenched by stirring with saturated NaHCO₃ aqueous solution for 2 hr or more. The aqueous phase is saturated with NaCl and extracted by DCM three times. The organic phase is washed by saturated NaHCO₃ aqueous solution three times, then water, brine, and dried by Na₂SO₄, filtered and evaporated to remove all volatiles. The residue can be dissolved in saturated NaHCO₃ aqueous solution. The aqueous phase can be saturated with NaCl and extracted by DCM three times. The organic phase is washed by saturated NaHCO₃ solution three times, then by water, brine, and dried by Na₂SO₄, filtered and evaporated to dryness to give the final product.

### Example 5C

### Attaching Pyrene Directly to Nanoparticle Comprising PEG Passivation Agent

The pyrene-NHS ester can be prepared as follows: To a round bottom flask with a stirring bar, 2-pyrenecarboxylic acid, N-hydroxysuccinimide (1.1 equiv.), DCC (1.1 equiv.) can be dissolved in DCM, capped and stirred at room temperature overnight. The mixture is filtered, washed by saturated NaHCO₃ aqueous solution three times and brine once, dried by Na₂SO₄, filtered and evaporated and subjected to Silica column using ethyl acetate (“EtOAc”) and hexane as eluent to yield the ester.

Pyrene can be attached to the nanoparticle as follows: To a round bottom flask with a stirring bar, nanoparticles comprising PEG passivation agent, 2-pyrene-carboxylic acid NHS ester (10 equiv.), DCM and triethylamine are added, and the flask is capped and stirred at room temperature for 3 days. Saturated NaHCO₃ aqueous solution is added and stirred at room temperature for a limited time to quench the reaction. More DCM would be added to extract nanoparticles from the reaction mixture. The DCM layer is washed by saturated NaHCO₃ aqueous solution three times and then by water and brine and dried by Na₂SO₄. The absence of NHS ester starting material and the presence of the pyrene group can be confirmed by ¹H NMR.

### Example 5D

### Attaching Anthracene Directly to Nanoparticle Comprising PEG Passivation Agent

The anthracene-carboxylic acid-NHS ester can be prepared as follows: To a round bottom flask with a stirring bar, 2-anthracenecarboxylic acid, N-hydroxysuccinimide (1.1 equiv.), and DCC (1.1 equiv.) are dissolved in DCM, and the flask is capped and stirred at room temperature overnight. The mixture is filtered, washed by saturated NaHCO₃ three times and brine once, dried by Na₂SO₄, filtered and evaporated and subjected to Silica column using EtOAc and hexane as eluent to yield the product.

Anthracene can be attached to the nanoparticle as follows: To a round bottom flask with a stirring bar, nanoparticles comprising PEG passivation agent, 2-anthracenecarboxylic acid NHS ester (10 equiv.), DCM and triethylamine are added, and the flask is capped and stirred at room temperature for 3 days. Saturated NaHCO₃ aqueous solution is added and stirred at room temperature for a limited time to quench the reaction. More DCM is added to extract nanoparticles from the reaction mixture. The DCM layer is washed by saturated NaHCO₃ aqueous solution three times and water, brine and dried by Na₂SO₄. The absence of NHS ester starting material and the presence of the anthracene group can be confirmed by ¹H NMR.

### Example 5E

### Attaching FITC Directly to Nanoparticle Comprising PEG Passivation Agent Not Using NHS Ester

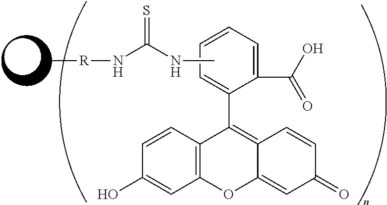

A solution of nanoparticles comprising PEG passivation agent in 0.1M sodium carbonate buffer, pH 9.5, was prepared at a concentration of around 2 mg/ml. FITC (a commercially-obtained composition containing isomers in which the isothiocyanate was attached at the 5- and 6-positions of the benzene ring) was freshly dissolved in DMF at a concentration of 2 mg/ml. The FITC solution was protected from light due to its low photo stability. The solution of nanoparticles comprising PEG passivation agent and the FITC/DMF solution were mixed in various molar ratio (5, 10, 20, 40 equiv. of Nanoparticles/PEG) in a round bottom flask with a stir bar. The mixture reacted under stirring at 4° C. in an ice bath for 4 hours and was kept in refrigerate at 4° C. for overnight. The FITC labeled derivative was purified by gel filtration (PD 10 column) using PBS buffer. The number of FITC per particle was determined by the absorption spectra with the known absorption coefficients of both FITC and Nanoparticles/PEG at 360 nm and 496 nm.

### Example 6

### Attaching Linker to Nanoparticle

It was demonstrated that attaching a linker to the passivation agent allows for a diverse array of chemistry with the nanoparticles of the present invention. As shown in the following reaction, so-called click chemistry is a facile method of connecting two species.

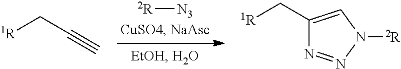

Either ¹R or ²R can be a nanoparticle with a passivation agent, a binding agent, a chromophore, a quencher, or other useful species. The alkynyl group (C≡C) and the azide group (N₃) represent linkers, and those linkers were attached to nanoparticles as follows.

### Example 6A

### Alkynylating Nanoparticles

4-pentynoic NHS ester was prepared as follows:

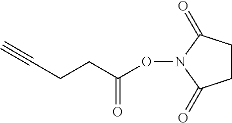

To a round bottom flask with a stirring bar, 4-pentynoic acid (1.0 g, 10 mmol), N-hydroxysuccinimide (1.3 g, 11 mmol), EDC (2.1 g, 11 mmol) were dissolved in dichloromethane (25 mL), capped and stirred at room temperature overnight. The mixture was washed by water three times and brine once, dried by Na₂SO₄, filtered and evaporated and subjected to silica column using EtOAc and hexane as eluent to give 1.1 g (57% yield) white solids. ¹H NMR (300 MHz, CDCl₃): δ=2.87 (2H, m, CH₂C═O), 2.83 (4H, m, O═CCH₂CH₂C═O), 2.61 (2H, m, CH₂C), 2.04 (1H, dd, CH).

Alkynylating the nanoparticles proceeded as follows:

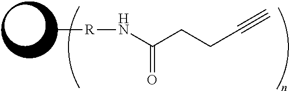

NaCl was added to nanoparticles comprising PEG passivation agent (0.180 g, 0.12 mmol) aqueous solution until it was saturated. DCM was added to extract the nanoparticles three times and dried by Na₂SO₄ for one hour or more. DCM solution was filtered and concentrated. To this solution, 4-pentynoic NHS ester (0.234 g 1.2 mmol) and triethylamine (0.3 mL) were added, capped and stirred at room temperature for 3 days. Saturated NaHCO₃ aqueous solution was added and stirred violently at room temperature for 24 h to quench the reaction. DCM was evaporated by rotorvap. The black mixture was diluted by water, extracted with EtOAc, saturated with NaCl, and extracted with DCM. The DCM layer was washed with saturated NaHCO₃ aqueous solution, water and brine, and dried by Na₂SO₄. After evaporation, it gave 0.176 g (93% yield). DMF can also be used as solvent to run the reaction. R in the structure above corresponds to the PEG passivation agent, and n is any suitable positive number. The variable n in the foregoing structure is limited by the number of amine groups found on the passivation agent molecules coupled to the nanoparticle.

### Example 6B

### Adding Azide Linker to Nanoparticles

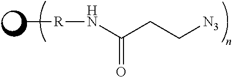

To a round bottom flask with a stirring bar, nanoparticles comprising PEG passivation agent (45 mg, 30E-6 mmol), 3-azidopropanoic NHS ester (63.6 mg, 300E-6 mmol) and TEA (20 mL) were dissolved in DCM (2 mL). After DCM was evaporated in 3 days, DMF (2 mL) and another batch of TEA (20 mL) were added and stirred for 54 h at 70° C. Saturated NaHCO₃ aqueous solution was added to quench the reaction and stirred for another 3 days. Water was added to dissolve the suspension and the solution was extracted with DCM. The organic layer was washed by saturated NaHCO₃ aqueous solution three times, water and brine and dried by Na₂SO₄. After evaporation of DCM, it gave about 40 mg black solids.

The synthesis can also be performed as follows: nanoparticles (1 equiv.), 3-azidopropanoic NHS ester (10 equiv.) and TEA (5% equiv.) will be dissolved in DMF (2 mL) and stirred for 3 days. Saturated NaHCO₃ aqueous solution will be added to quench the reaction and stirred for another 1 day. Water will be added to dissolve the suspension and the solution will be extracted with DCM. The organic layer will be washed by saturated NaHCO₃ aqueous solution three times, water and brine and dried by Na₂SO₄. After evaporation of DCM, it will give black solids as the product.

### Example 7

### Attaching Binding Agent to Nanoparticle Via Linker

### Example 7A

### Biotin Attached to Nanoparticle Via Alkyne Linker

Biotin azide was prepared as follows:

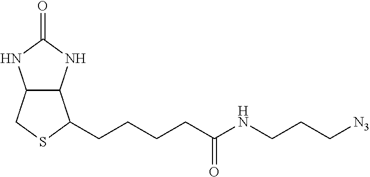

To a round bottom flask with a stirring bar, biotin NHS ester (1.0 g, 2.9 mmol) was dissolved in DMF (30 mL), followed by addition of 3-azidopropylamine (0.33 g, 3.3 mmol) and TEA (0.61 mL). The mixture was stirred at room temperature overnight. Most DMF was removed by rotorvap. The residue solution was precipitated in ether and recrystallize from isopropanol to give 0.52 g (55%) white solids after drying in vacuo. ¹H NMR (300 MHz, DMSO-d₆): δ=7.83 (1H, t, NHCH₂), 6.40 (1H, s, NH), 6.34 (1H, s, NH), 4.28 (1H, m, bridge CH), 4.12 (1H, m, bridge CH), 3.08 (3H, m, thiophene CH), 2.81 (1H, m, thiophene CH₂), 2.57 (1H, m, CHC), 2.06 (2H, t, CH₂N), 1.63-1.42 (6H, m, CH₂CH₂CH₂), 1.28 (2H, m, CH₂CH₂N₃).

Starting reagent 3-azidopropyl amine can be synthesized as described in Knoer et al., Chemistry—A European Journal (2007), 13(21), 6082-6090, S6082/1-S6082/20.

Nanoparticle/Peg/Alkyne Linker reacted with Biotin Azide as follows:

To a round bottom flask with a stirring bar, alkynylated nanoparticles comprising PEG passivation agent, biotinylated azide (5 equiv.), CuSO₄.5H₂O (5 equiv.) were dissolved in DMSO (1 mL) and water (0.1 mL). Sodium ascorbate (10 equiv.) was added. The mixture was stirred at room temperature and became brown soon. After 42 hours, the reaction was quenched by addition of water. It was extracted by EtOAc three times, then acidified by diluted HCl solution and extracted with brine and DCM three times. The DCM layer was combined and washed with brine and dried by Na₂SO₄, filtered and evaporated to give the product. NMR confirmed the attachment of the alkyne linker to the nanoparticle, but the signal to noise ratio was not optimal.

### Example 7B

### Biotin Attached to Nanoparticle Via Azide Linker

Biotin Alkyne was prepared as follows:

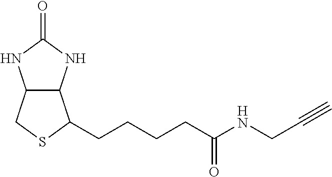

To a round bottom flask with a stirring bar, biotin NHS ester (0.46 g, 1.35E-6 mol) was dissolved in DMF (20 mL), followed by addition of propargylamine (0.083 g, 1.5E-6 mol) and TEA (0.02 mL). The mixture was stirred at room temperature overnight. Most DMF was removed by rotorvap. The residue solution was precipitated in ether and recrystallize from isopropanol to give 0.166 g (43%) off-white solids after drying in vacuo. ¹H NMR (300 MHz, DMSO-d₆): δ=8.20 (1H, t, NHCH₂), 6.40 (1H, s, NH), 6.34 (1H, s, NH), 4.28 (1H, m, bridge CH), 4.11 (1H, m, bridge CH), 3.80 (2H, m, CH₂NH), 3.06 (2H, m, thiophene CH), 2.81 (1H, m, thiophene CH₂), 2.57 (1H, m, CH), 2.06 (2H, t, CH₂C═O), 1.51 (4H, m, CH₂CH₂), 1.30 (2H, m, CH₂).

Nanoparticle/PEG/Azide Linker can be reacted with Biotin Alkyne as follows:

Nanoparticles comprising PEG passivation agent and the azide linker (1 equiv.) and biotin alkyne (1 equiv.) will be dissolved in suitable solvent for example DMF. CuSO₄.5H₂O (1 equiv.) and sodium ascorbate (1 equiv.) will be added to the mixture and stirred for 3 days at rt. Brine will be added. The mixture will be extracted with n-butanol three times, EtOAc three times and DCM three times. The DCM layer will be washed by brine and dried by Na₂SO₄. After evaporation of DCM, it will give black solids as the product.

### Example 7C

### Folate Azide

Folate azide was prepared as follows:

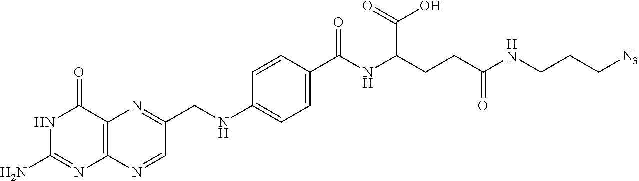

Anhydrous folic acid (1.0 g, 2.3 mmol) was dissolved in DMSO (9 mL), followed by addition of 3-azidopropylamine (0.240 g, 2.3 mmol), DCC (1.168 g, 5.7 mmol) and pyridine (4.5 mL). The mixture was stirred at room temperature for 24 h. It was filtered and precipitated in cold ether. The precipitates were collected by centrifugation. The sediment was washed and centrifuged, and the process was repeated three times. After drying in vacuo, it gave 1.16 g yellow solids (98% yield). ¹H NMR (300 MHz, d₆-DMSO): δ=11.45 (1H, br, COOH), 8.62 (1H, m, Ar—H), 8.12 (1H, m, NH), 7.94 (1H, m, NH), 7.88 (1H, m, NH), 7.62 (2H, m, NH₂), 6.90 (2H, m, Ar—H), 6.62 (2H, m, Ar—H), 4.47 (2H, m, CH₂N), 4.27 (1H, m, CH), 3.87 (1H, br, NH), 3.09 (2H, t, CH₂NH), 2.24 (2H, m, CH₂), 1.91 (2H, m, CH₂), 1.61 (4H, m, CH₂CH₂N₃). Folate azide was attached to nanoparticles having an alkynyl linker analogously to the reaction described for biotin azide in Example 7A.

### Example 8

### Addition of Linkers to Binding Agents and Chromophores to Control Attachment and Hydrophilicity

To modify the hydrophilicity of the nanoparticle complexes, and to control the distance between the nanoparticle and the binding agents and chromophores attached to the nanoparticle, additional linker molecules were synthesized as follows. Then, these linker molecules were attached either to nanoparticles via (a) the amine of the passivation agent, (b) the alkynyl linker attached to the passivation agent, or (c) the azide linker attached to the passivation agent as described above; or these linker molecules were attached to binding agents or chromophores, and then attached to the nanoparticles. Some of the chromophores below have the azide or alkynyl linker only.

### Example 8A

### 1,11-diazido-3,6,9-trioxaundecane

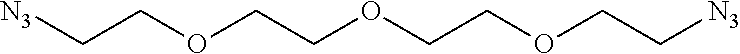

Tetraethylene glycol (20.0 g, 0.103 mol) and TEA (24.2 mL, 0.174 mol) were dissolved in THF, followed by addition of mesyl chloride (“MsCl”) (17.6 mL, 0.227 mol) dropwise at 0° C. It was stirred overnight. NaN₃ (20.1 g, 0.309 mol) and water (1 mL) were added. The mixture was heated to reflux overnight. After concentration of the solution under reduced pressure, it gave 18.37 g yellow sticky liquid. The yield of the two steps above was 73%. ¹H NMR (300 MHz, CDCl₃): δ=3.74-3.56 (12H, m, CH₂O), 3.35 (4H, t, CH₂N₃).

### Example 8B

### 1-amino-11-azido-3,6,9-trioxaundecane

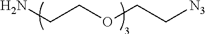

Triphenyl phosphine (1.29 g, 5 mmol) was dissolved in ether (10 mL) and added to the solution of 1,11-diazido-3,6,9-trioxaundecane (1.20 g, 5 mmol) in ether/THF/HCl 1M (18 mL, 4/1/4) at room temperature dropwise. The mixture was stirred violently overnight when HCl (4 M) was added to extract the pale brown mixture twice. The aqueous layer was extracted with EtOAc three times. After its pH was brought to 14 by NaOH pellet in ice water bath, ether was added to extract the solution twice. The aqueous layer was extracted by DCM three times. The organic phase was combined and dried by Na₂SO₄. After evaporation, it gave 0.26 g oil (23% yield). ¹H NMR (300 MHz, CDCl₃): δ=5.79 (2H, br, NH₂). 3.61 (12H, m, CH₂C—O), 3.36 (1H, t, CH₂C), 2.98 (3H, m, CH₂CH).

### Example 8C

### Hydrophilic Biotin-Azide

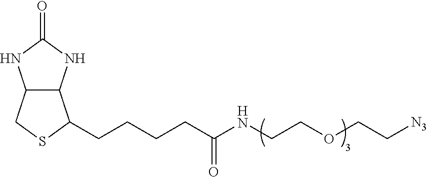

Biotin NHS ester (0.474 g, 1.4 mmol) was dissolved in DMF (10 mL), followed by addition of 1-amino-11-azido-3,6,9-trioxaundecane (0.380 g, 1.7 mmol) and TEA (0.3 mL). The mixture was stirred at room temperature overnight. It was diluted with EtOAc, washed with HCl, brine and dried by Na₂SO₄. After evaporation, it was subjected to column to give 0.405 g white solids (66% yield). ¹H NMR (300 MHz, CDCl₃): δ=6.77 (1H, s, NH), 6.34 (1H, s, NH), 4.49 (1H, m, bridge CH), 4.33 (1H, m, bridge CH), 3.65 (11H, m, OCH₂), 3.40 (4H, m, OCH₂), 3.12 (1H, m, thiophene CH), 2.88 (1H, m, thiophene CH₂), 2.76 (1H, m, thiophene CH₂), 2.22 (2H, t, CH₂N), 1.68 (4H, m, CH₂CH₂), 1.43 (2H, m, CH₂), 1.18 (2H, m, CH₂CH₂CH₂).

### Example 8D

### Hydrophilic Fluorescein-Azide

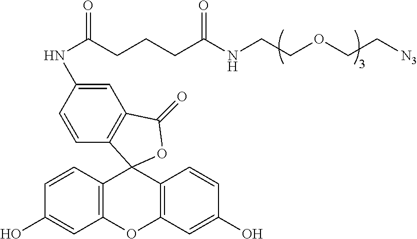

Fluorescein pentanoic acid (0.23 g, 0.5 mmol) and NHS (0.069 g, 0.6 mmol) were dissolved in DMF (6 mL) and DCM (2 mL), followed by addition of EDC (0.115 g, 0.6 mmol). The mixture was stirred overnight. The linker 1-amino-11-azido-3,6,9-trioxaundecane (0.137 g, 0.6 mmol) and triethylamine (0.137 mL, 0.6 mmol) were added. The mixture was stirred at room temperature overnight. The mixture was precipitated in water. After centrifugation, the sediment was collected and dried and subjected to silica column to give 0.14 g deep red solids (37% yield). ¹H NMR (300 MHz, DMSO-d₆): δ=10.34 (1H, br, COOH), 10.09 (2H, br, OH), 8.31 (1H, s, NH), 7.89 (1H, t, NH), 7.81 (1H, m, Ar—H), 7.17 (1H, d, Ar—H), 6.65 (2H, m, Ar—H), 6.57 (5H, m, Ar—H), 3.50-3.57 (14H, m, OCH₂CH₂), 3.20 (2H, m, CH₂N₃), 2.36 (2H, t, CH₂C═O), 2.14 (2H, t, CH₂C═O), 1.82 (2H, m, CH₂CH₂CH₂).

Fluorescein pentanoic acid was synthesized as described in Gross et al., J. Am. Chem. Soc., (2005), 127(42), 14588-14589.

### Example 8E

### Hydrophilic Rhodamine-Azide

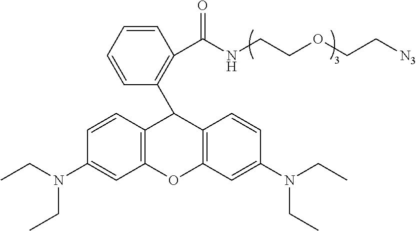

Rhodamine B (0.313 g, 0.655 mmol), EDC (0.169 g, 0.88 mmol) and the linker 1-amino-11-azido-3,6,9-trioxaundecane (0.100 mg, 0.439 mmol) were dissolved in DCM (10 mL), followed by addition of diisopropylethylamine (“DIPEA”) (0.3 mL) and catalytic amount of 4-dimethylaminopyridine (“DMAP”) (5 mg). The pink mixture was stirred overnight. The mixture was stirred at room temperature overnight. The mixture was concentrated and subjected to silica column to give 0.2 g red oil (65% yield). ¹H NMR (300 MHz, CDCl₃): δ=7.88 (1H, m, Ar—H), 7.41 (2H, m, Ar—H), 7.06 (1H, m, Ar—H), 6.41 (2H, m, Ar—H), 6.36 (2H, m, Ar—H), 6.27 (2H, m, Ar—H), 3.59-3.62 (4H, m, CH₂C—O), 3.49 (2H, t, CH₂C), 3.27-3.40 (14H, m, CH₂CH₃+CH₂CH), 3.16 (2H, t, CH₂NH), 1.78 (2H, s, CH₂N₃), 1.18 (12H, t, CH₃).

### Example 8F

### Fluorecein-Azide

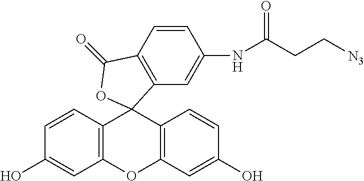

The reagent 6-aminofluorecein (0.3 g, 0.86 mmol) was dissolved in DMF (7 mL) followed by addition of 3-azidoproponic NHS ester (0.945 g, 4.3 mmol) and triethylamine (0.2 mL). The mixture was stirred at room temperature for two days. The mixture was precipitated in water. After centrifugation, the sediment was collected and dried to give 0.21 g yellow solids (55% yield). ¹H NMR (300 MHz, DMSO-d₆): δ=10.04 (1H, br, COOH), 7.55 (1H, d, Ar—H), 6.73 (2H, d, Ar—H), 6.60 (3H, m, Ar—H), 6.55 (2H, m, Ar—H), 6.23 (1H, br, NH), 6.07 (1H, m, Ar—H), 2.83 (2H, m, CH₂C═O), 2.64 (2H, m, CH₂N₃).

### Example 8G

### Hydrophilic Folate with Alkynyl Linker

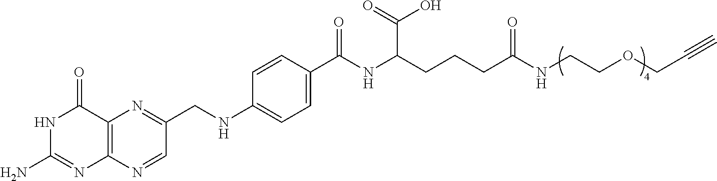

Anhydrous folic acid (1 equiv.) can be dissolved in DMSO, followed by addition of 3,6,9,12-tetraoxapentadec-14-yn-1-ol, EDC (1.1 equiv.), HOBt (2 equiv.) and TEA (1 equiv.). The mixture would be stirred at room temperature for 24 h. It will be filtered and precipitated in cold ether. The precipitates can be collected by centrifugation. The sediment will be washed by ether and centrifuged and the process will be repeated three times. After drying under vacuo, it will give the final product.

3,6,9,12-Tetraoxapentadec-14-yn-1-ol can be prepared according to Polito et al., Chemical Communications (2008), (5), 621-623.

### Example 8H

### 3-Azido-7-Hydroxy-Coumarin, “Coumarin Azide”

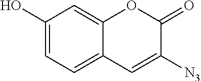

Compound 3-azido-7-hydroxy-coumarin was prepared following the procedure set forth in Sivakumar et al., Org. Lett. 2004, 6, 4603-4606.

### Example 81

### 3-Azidoproponic NHS Ester

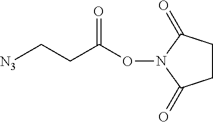

The synthesis began with the reaction of 3-bromoproponic acid with NaN₃ with 26% yield due to the good solubility of resultant product in water, followed by the reaction with NHS activated by EDC to give 3-azidoproponic NHS ester with 97% yield, for a 25% total yield in two steps. ¹H NMR (300 MHz, CDCl₃): δ=3.68 (2H, t, CH₂C═O), 2.89 (2H, t, CH₂N₃), 2.85 (4H, s, CH₂C═O).

### Example 8J

### Click Reaction of 4-Pentynoic Acid with Coumarin Azide

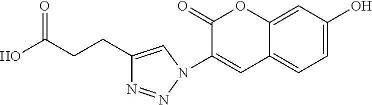

To a round bottom flask with a stirring bar, 4-pentynoic acid (24.1 mg, 0.25E-6 mol), coumarin azide (50 mg, 0.25E-6 mol) and CuSO₄.5H₂O (6.2 mg, 0.25E-7 mol) were mixed in DMSO (1 mL) and water (0.1 mL). Sodium ascorbate (9.8 mg, 0.49E-7 mol) was added. The mixture became brown soon and was stirred at room temperature. After 42 hours, the reaction was quenched by addition of water. It was extracted by EtOAc three times, then acidified by diluted HCl solution and extracted by brine and DCM three times. DCM layer was combined and washed with brine and dried by Na₂SO₄, filtered and evaporated to give yellow solids. After washed with EtOAc, it gave 25.0 mg (33% yield) yellow solids. ¹H NMR (300 MHz, CDCl₃): δ=12.21 (1H, br, COOH), 10.86 (1H, s, OH), 8.54 (1H, s, Ar—H), 8.31 (1H, s, Ar—H), 7.73 (1H, d, Ar—H), 6.88 (1H, dd, Ar—H), 6.83 (1H, s, triazole-H), 2.93 (2H, m, CH₂), 2.63 (2H, m, CH₂). This molecule is useful as a biomarker.

### Example 9

### Attaching Chromophore to Nanoparticle Via Linker

### Example 9A

### Nanoparticle/PEG/Coumarin Via Alkynyl Linker

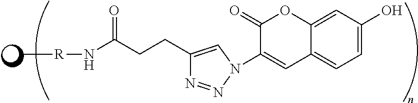

To a round bottom flask with a stirring bar, nanoparticles comprising PEG and the alkyne linker (15.0 mg, 15E-6 mol), 3-azido-7-hydroxy-coumarin (3.1 mg, 15E-6 mol) and CuSO₄.5H₂O (3.8 mg, 15E-6 mol) were mixed in DMSO (1 mL) and water (0.1 mL). Sodium ascorbate (3 mg, 15E-6 mol) was added. The mixture became brown soon and was stirred at room temperature. After 42 hours, the reaction was quenched by addition of water. It was extracted by EtOAc three times, then acidified by diluted HCl solution and extracted by brine and DCM three times. DCM layer was combined and washed with brine and dried by Na₂SO₄, filtered and evaporated to give 15 mg product (84% yield).

As one of ordinary skill in the art can appreciate, additional chromophores can attach to a nanoparticle using analogous chemistry.

### Example 10

### Attaching Binding Agent and Chromophore to Nanoparticle Via Linker

Several “dual click” reactions were performed, wherein the nanoparticle comprising PEG passivation agent with the alkynyl linker were reacted with binding agents and chromophores, both of which had azide groups to “click” with the alkyne. Thus, nanoparticles were prepared that had both binding agent and chromophore attached. Those dual-functionalized nanoparticles were formed as follows:

(a) Nanoparticle/PEG/alkyne (0.010 g, 6.3E-3 mmol) and (b) folate-azide (0.95, 0.70, 0.50, 0.25, 0.10 and 0 equiv.) or biotin-azide and (c) coumarin azide or fluorescein azide or rhodamine azide (0.05, 0.30, 0.50, 0.75, 0.90 and 1 equiv.), respectively were dissolved in DMSO (1 mL), followed by addition of CuSO₄.5H₂O (5 mg, 20.0E-3 mmol), sodium ascorbate (12 mg, 86.8E-3 mmol) and water (0.05 mL). The mixture turned deep brown and was stirred at room temperature. After more than 48 h, the reaction mixture was saturated with NaCl, and extracted with butanol. The butanol layer was washed with H₂O. The aqueous layer was washed by butanol three times or more until butanol layer was colorless, then EtOAc three times to give final solution.

### Example 10A

### Nanoparticles/PEG/Alkynyl Linker/(Biotin and Coumarin)

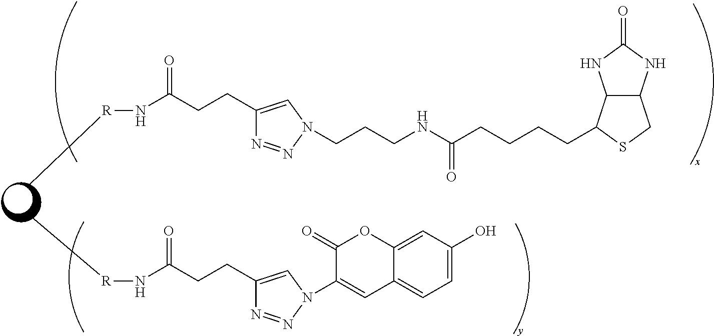

The biotin azide and coumarin azide were mixed in various ratios to one equivalent of alkyne (which was calculated based on the passivating agent molecular weight and the mass of the nanoparticle, assuming one alkyne to each molecule of passivation agent) on nanoparticles comprising PEG passivation agent, which underwent click reaction catalyzed by Cu(I) in situ prepared via reduction of CuSO₄ by sodium ascorbate to give dual modified nanoparticles. The numbers of alkyne on the nanoparticle were estimated based on the maximum number of passivation agent molecules measured or calculated to be present on the nanoparticle, since each passivation agent molecule possessed a single site (an amine) onto which the alkyne could form. That assumes that di-amino terminated passivating agents couple with one amine to the carbon core, leaving the second amine free to form the alkyne. Table I summarizes biotinylations under various conditions, including versus different ratios of coumarin.

TABLE I

| | Designed | | | | |
|---|---|---|---|---|---|
| | percentage of | | | | |
| | Coumarin- | | Biotin per | Coumarin | Quantum |
| Methods | N₃/Biotin-N₃ | | Nanoparticle | number | yield |
| Click chemistry | 100% | 0% | 0 | 220 | 6.8% |
| (CuSO₄, | 75% | 25% | 0 | 144 | 5.6% |
| sodium | 50% | 50% | 3.8 | 144 | 4.9% |
| ascorbate, | 25% | 75% | 9.4 | 165 | 3.6% |
| DMSO, H₂O) | 10% | 90% | 1.9 | 82 | 2.3% |
| | 0% | 100% | 1.8 | N/A | 1.4% |
| DMF, PBS, | biotin | rt/70° C. | 1.8-2.7 | N/A | N/A |
| NaHCO₃ | NHS | | | | |
| DMF, TEA | biotin | rt/70° C. | 0.42-5.8 | N/A | N/A |
| | NHS | | | | |

To test the available number of biotin per nanoparticle, biotin-containing nanoparticles were subjected to quantitative assay with a biotin quantification assay. Specifically, when 4′-hydroxyazobenzene-2-carboxylic acid (HABA) is mixed with avidin, it forms a complex that is a yellow chromophore with molar extinction coefficient 34000 M−1·cm−1 at 500 nm. However, biotin has much higher binding coefficient towards avidin than that of HABA. When biotin conjugated materials are mixed with the HABA-avidin complex, biotin replaces HABA to give a biotin-avidin complex that barely absorbs light at 500 nm. Thus, the chromophore concentration decreases. By measuring the absorbance difference at 500 nm using UV-Vis spectroscopy, the number of avidin-accessible biotin can be calculated.

The coumarin number was determined by comparing the UV-Vis spectrum of the coumarin bound to the nanoparticle with that of the coumarin derivative set forth in Example 8J in a composition having a known concentration. Dividing the measured concentration of the bound coumarin by the concentration of nanoparticles yields the number of coumarin per nanoparticle. The quantum yield was measured by using coumarin-1 as a standard according to conventional methods.

### Example 10B

### Nanoparticle/PEG/Alkynyl Linker/(Hydrophilic Biotin and Coumarin)

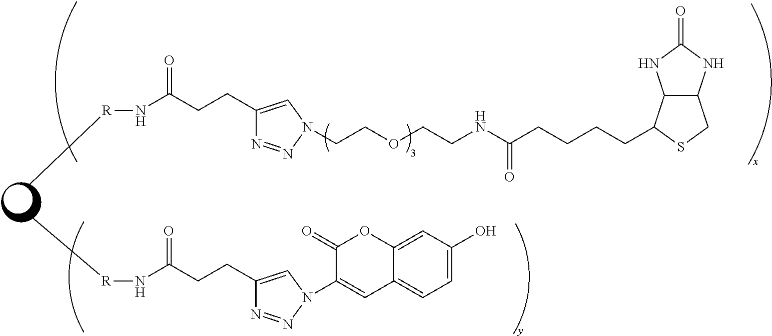

Hydrophilic biotin azide and coumarin azide were reacted in various ratios with nanoparticles comprising PEG and alkynyl linker. Table II shows the synthetic yield, quantum yield, and measured biotin per nanoparticle for various ratios of coumarin azide to hydrophilic biotin azide.

TABLE II

| Reaction loading | | | | |
|---|---|---|---|---|
| percentage of | | | Quantum yield | Biotin per |
| Coumarin-N₃/Biotin-N₃ | | Yield | (λₑₓ360 nm) | Nanoparticle |
| 0% | 0% | N/A | 1.9% | N/A |
| 0% | 100% | 45% | N/A | 9.6 |
| 5% | 95% | 82% | 6.1% | 6.9 |
| 30% | 70% | 97% | 17.3% | 4.9 |
| 50% | 50% | 99% | 16.7% | 2.3 |
| 75% | 25% | 100% | 17.0% | 1.5 |
| 90% | 10% | 84% | 19.0% | −0.4 |
| 100% | 0% | 98% | 16.6% | 0 |

The foregoing data suggest that several suitable ratios of coumarin to biotin exist, such as, for example, 30% coumarin to 70% biotin.

### Example 10C

### Nanoparticle/PEG/Alkynyl Linker/(Hydrophilic Biotin and Hydrophilic Fluorescein)

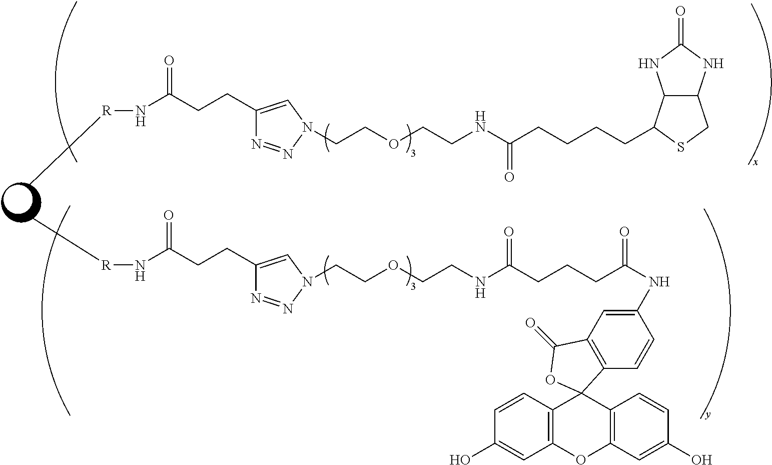

Hydrophilic biotin and hydrophilic fluorescein were reacted in various ratios with nanoparticles comprising PEG passivation agent and the alkynyl linker. Table III shows the synthetic yield, fluorescence quantum yield, and measured biotin per nanoparticle for various ratios of hydrophilic fluorescein to hydrophilic biotin.

TABLE III

| Reaction loading percentage of | | | | |
|---|---|---|---|---|
| Hydrophilic Fluorescein-N₃/ | | | Quantum yield | Biotin per |
| Hydrophilic Biotin-N₃ | | Yield | (λₑₓ470 nm) | Nanoparticles |
| 0% | 0% | N/A | 1.8% | N/A |
| 0% | 100% | 45% | N/A | 9.6 |
| 5% | 95% | 96% | 41.9% | 7.5 |
| 30% | 70% | 86% | 71.4% | 5.4 |
| 50% | 50% | 64% | 72.6% | 6.1 |
| 75% | 25% | 57% | 68.0% | 3.2 |
| 90% | 10% | 40% | 70.3% | 3.6 |
| 100% | 0% | 29% | 74.2% | 0 |

The foregoing data suggest that several suitable ratios of hydrophilic fluorescein to hydrophilic biotin exist for imaging cancer cells, among other uses, such as, for example, 30% hydrophilic fluorescein to 70% hydrophilic biotin.

### Example 10D

### Nanoparticle/PEG/Alkynyl Linker/(Folate and Coumarin)

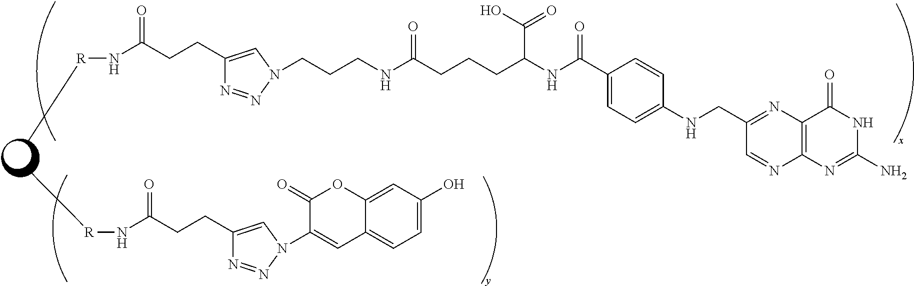

Folate azide and coumarin azide were reacted in various ratios with nanoparticles comprising PEG passivation agent and the alkynyl linker. Table IV shows the synthetic yield and fluorescence quantum yield for various ratios of coumarin to folate.

TABLE IV

| | | |
|---|---|---|
| Designed percentage of | | Quantum yield |
| Coumarin-N₃/folate-N₃ | Yield | (λₑₓ360 nm) |

| | | | |
|---|---|---|---|
| 5% | 95% | 42% | 5.0% |
| 30% | 70% | 68% | 6.1% |
| 50% | 50% | 89% | 8.8% |
| 75% | 25% | 90% | 15.1% |
| 90% | 10% | 90% | 17.0% |
| 100% | 0% | 33% | 16.4% |

The foregoing data suggest that several suitable ratios of coumarin to folate exist for imaging cancer cells, among other uses, such as, for example, 90% coumarin to 10% folate.

### Example 10E

### Nanoparticle/ED/Alkynyl Linker/(Folate and Coumarin)

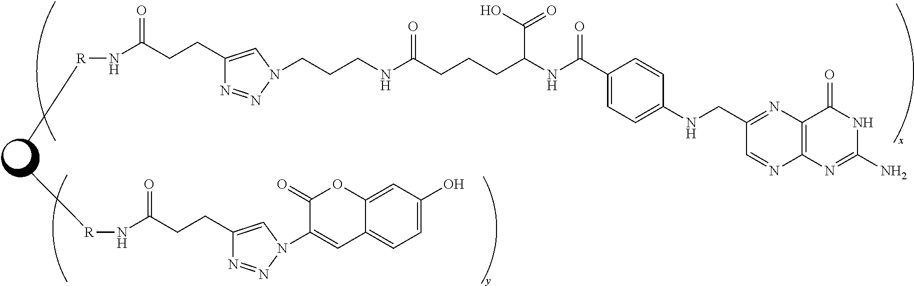

Folate azide and coumarin azide were reacted in various ratios with nanoparticles comprising ED passivation agent and the alkynyl linker. R in the foregoing molecular structure relates to diamine terminated oligomeric poly(ethylene glycol) passivation agent described in Example 2. Table V shows the synthetic yield and fluorescence quantum yield for various ratios of coumarin to folate.

TABLE V

| | | |
|---|---|---|
| Designed percentage of | | Quantum yield |
| Coumarin-N₃/folate-N₃ | Yield | (λₑₓ360 nm) |

| | | | |
|---|---|---|---|
| 0% | 0% | N/A | 1.2% |
| 5% | 95% | 8% | 10.9% |
| 30% | 70% | 13% | 13.8% |
| 50% | 50% | 22% | 10.2% |
| 75% | 25% | 26% | 28.3% |
| 90% | 10% | 38% | 16.4% |
| 100% | 0% | 30% | 25.3% |

The foregoing data suggest that several suitable ratios of coumarin to folate exist for imaging cancer cells, among other uses, such as, for example, 75% coumarin to 25% folate.

### Example 10F

### Nanoparticle/[PEG or ED]/Alkynyl Linker/(Folate and Hydrophilic Fluorescein)

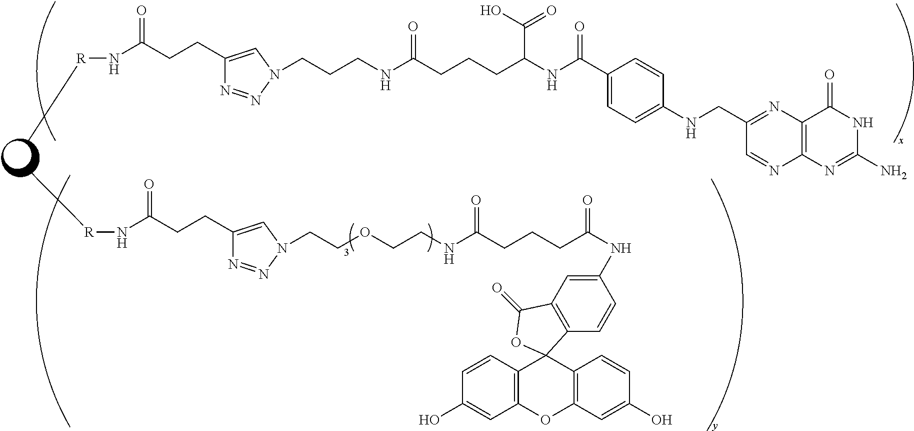

R in the foregoing structure is PEG or ED, as both markers were synthesized. x and y are any suitable positive numbers. In some cases, the sum of x and y is limited by the number of free amine groups of the passivation agent on the nanoparticle. Folate azide and hydrophilic fluorescein azide were reacted in various ratios with nanoparticles comprising the PEG passivation agent or the ED passivation agent and the alkynyl linker. Table VI describes the synthetic yield and fluorescence quantum yield for various ratios of folate to hydrophilic fluorescein on nanoparticles comprising PEG passivation agent.

TABLE VI

| | | |
|---|---|---|
| Reaction loading percentage of | | Quantum yield |
| fluorescein-N₃/folate-N₃ | Yield | (λₑₓ470 nm) |

| | | | |
|---|---|---|---|
| 0% | 0% | N/A | 1% |
| 0% | 100% | 45% | 1% |
| 5% | 95% | 100% | 4.8% |
| 30% | 70% | 79% | 29% |
| 50% | 50% | 75% | 42% |
| 75% | 25% | 86% | 79% |
| 90% | 10% | 73% | 59% |
| 100% | 0% | 49% | 48% |

Fluorescein quantum yield was measured using fluorescein as standard. All sample concentrations were adjusted to have absorption value of 0.08 at 470 nm at which they were excited for fluorescence.

Table VII describes the synthetic yield and fluorescence quantum yield for various ratios of folate to hydrophilic fluorescein on nanoparticles comprising ED passivation agent.

TABLE VII

| | | |
|---|---|---|
| | | Quantum |
| Reaction loading percentage of | | yield |
| Fluorescein-N₃/folate-N₃ | Yield | (λₑₓ470 nm) |

| | | | |
|---|---|---|---|
| 0% | 0% | N/A | 1% |
| 5% | 95% | 5% | N/A |
| 30% | 70% | 20% | 42% |
| 50% | 50% | 28% | 4% |
| 75% | 25% | 18% | 58% |
| 90% | 10% | 9% | 72% |
| 100% | 0% | 1% | 76% |

These results suggest many suitable ratios of hydrophilic fluorescein to folate on nanoparticles, such as, for example, 75% hydrophilic fluorescein to 25% folate on PEG-coated nanoparticles, and 90% hydrophilic fluorescein to 10% folate on ED-coated nanoparticles.

Initial attempts to synthesize folate-fluorescein and hydrophilic biotin-rhodamine B on nanoparticles comprising PEG passivation agent and alkynyl linker were not successful yet.

### Example 10G

### Nanoparticle/PEG/(FITC and Biotin) Using Two Different Chemistries

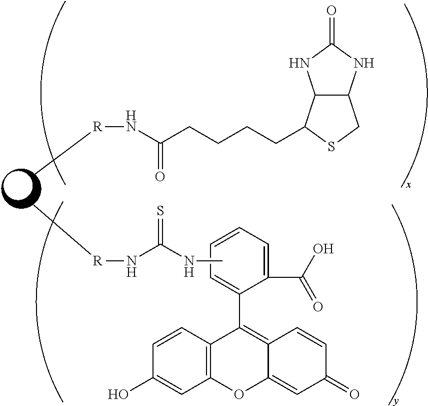

Biotin-NHS was dissolved in DMF at a concentration of 10 mM. The purified Nanoparticles/PEG/FITC in PBS buffer solution was mixed with the Biotin-NHS/DMF (50 equiv. of Nanoparticles/PEG/FITC as described in Example 5E) in a round bottom flask with a stir bar. The mixture reacted under stirring at room temperature for 0.5 hour. The dual functionalized Nanoparticles/PEG/FITC/Biotin was purified by gel filtration (PD 10 column) using PBS buffer. The number of biotin per particle was determined by the HABA assay kit described in Example 10A.

To test the limit of detection, a streptavidin binding assay was conducted with nanoparticles comprising PEG passivation agent, biotin binding agent, and FITC chromophore. A streptavidin coated well plate was incubated with varying concentrations of those nanoparticles and then washed to remove the unbound particles. The fluorescence of the resulting complex at different concentrations was then measured using a GENios Plate Reader (485 nm Ex/535 nm Em) with the gain set by the intensity of the brightest well. It was determined that the limit of detection was about 1 nM (one nanomolar) concentration for those nanoparticles.

As one of ordinary skill in the art can appreciate, more than one chromophore, more than one binding agent, and combinations thereof can be attached to a nanoparticle through analogous chemistry. For example, a nanoparticle/linker can be simultaneously exposed to more than one chromophore, wherein each chromophore has an active group that reacts with the linker. By modifying the ratios of the several chromophores present in the reaction vessel, markers comprising various ratios of chromophores can be formed.

### Example 11

### Chromophore-Binding Agent Pairs

For various purposes, chromophore-binding agent pairs were synthesized. Some pairs employed alkyne-azide click chemistry, while other pairs also included a hydrophilic linker. Among other uses, these molecules can act as luminescent markers for various biomedical applications. Accordingly, some embodiments of the present invention relate to methods of investigating at least one analyte, comprising: correlating the at least one analyte with at least one marker; and observing the luminescent emission of the at least one marker; wherein the at least one marker comprises at least one chromophore covalently coupled to at least one binding agent, optionally further comprising at least one linker covalently coupling the at least one chromophore and at least one binding agent. Further embodiments of the present invention relate to markers comprising at least one chromophore covalently coupled to at least one binding agent, optionally further comprising at least one linker covalently coupling the at least one chromophore and at least one binding agent.

### Example 11A

### Click Reaction of Biotin Alkyne with Coumarin Azide

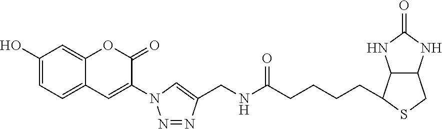

To a round bottom flask with a stirring bar, biotin alkyne (69.2 mg, 0.25E-6 mol), coumarin azide (50 mg, 0.25E-6 mol) and CuSO₄.5H₂O (61.6 mg, 0.25E-6 mol) were mixed in DMSO (1 mL) and water (0.1 mL). Sodium ascorbate (48.7 mg, 0.25E-6 mol) was added. The mixture became brown soon and was stirred at room temperature. After three days, the reaction was quenched by addition of HCl solution (1 M). The white precipitate was filtered, washed with water and dried to give 100.0 mg (84% yield) white solids. ¹H NMR (300 MHz, DMSO-d₆): δ=10.90 (1H, br, OH), 8.56 (1H, s, Ar—H), 8.39 (1H, t, NH), 8.33 (1H, s, Ar—H), 7.74 (1H, d, Ar—H), 6.91 (1H, d, Ar—H), 6.84 (1H, s, triazole-H), 6.39 (1H, s, NH), 6.32 (1H, s, NH), 4.35 (2H, m, CH₂NH), 4.27 (1H, m, bridge CH), 4.09 (1H, m, bridge CH), 3.07 (2H, m, thiophene CH), 2.76 (1H, m, thiophene CH₂), 2.54 (1H, m, CH), 2.10 (2H, t, CH₂C═O), 1.50 (4H, m, CH₂CH₂), 1.29 (2H, m, CH₂).

### Example 11B

### Hydrophilic Biotin-Fluorescein

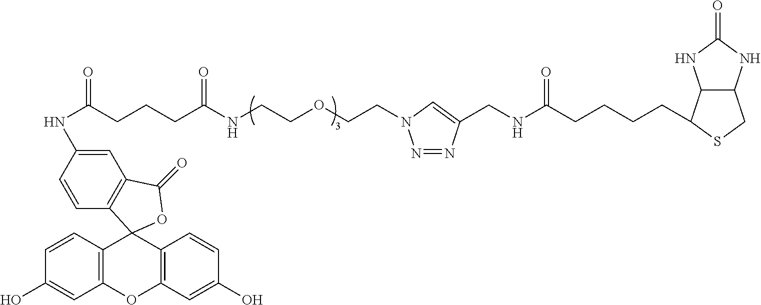

Biotin alkyne (0.010 g, 35.6 E-6 mol) and hydrophilic fluorescein azide (23.9 mg, 35.6E-6 mol) were dissolved in DMSO (1 mL), followed by addition of CuSO₄.5H₂O (10 mg, 40.0E-6 mol), sodium ascorbate (20 mg, 104E-6 mol) and water (0.1 mL). The mixture turned deep brown and was stirred at room temperature. After 48 hours, the reaction mixture was centrifuged and the supernatant was precipitated by water and centrifuged to collect the sediment. It was washed by DCM and dried. It gave 32 mg (94%) deep red solids. ¹H NMR (300 MHz, DMSO-d₆): δ=10.34 (1H, br, COOH), 10.09 (2H, br, OH), 8.30 (1H, m, NH), 8.26 (1H, m, NH), 7.88 (1H, t, Ar—H), 7.80 (1H, s, triazole-H), 7.19 (1H, d, Ar—H), 6.64 (2H, m, Ar—H), 6.55 (3H, m, Ar—H), 6.40 (1H, s, NH), 6.34 (1H, s, NH), 4.46 (2H, m, CH₂N), 4.26 (1H, m, bridge CH), 4.11 (1H, m, bridge CH), 3.77 (2H, m, OCH₂CH₂N), 3.46 (8H, br, OCH₂), 3.38 (6H, m, OCH₂CH₂O), 3.18 (2H, m, CH₂N), 3.06 (1H, m, thiophene CH), 2.81 (1H, m, thiophene CH₂), 2.76 (1H, m, thiophene CH₂), 2.35 (2H, m, CH₂C═O), 2.14 (2H, m, CH₂C═O), 1.81 (2H, m, CH₂CH₂CH₂), 1.50 (4H, m, CH₂CH₂), 1.26 (2H, m, CH₂).

### Example 11C

### Hydrophilic Folate-Coumarin

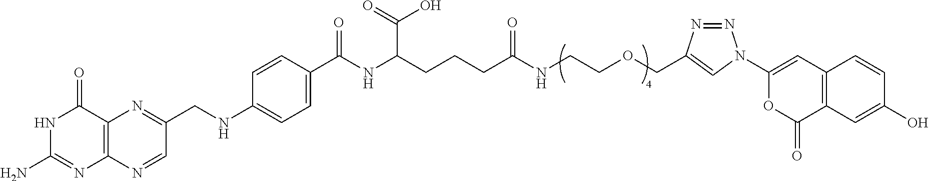

Hydrophilic folate alkyne (0.020 g, 30.5E-6 mol) and coumarin azide (6.2 mg, 30.5E-6 mol) were dissolved in DMSO, followed by addition of CuSO₄.5H₂O (8 mg, 32.0E-6 mol), sodium ascorbate (12 mg, 62.5E-6 mol) and water (0.05 mL). The mixture turned deep brown and was stirred at room temperature. After 48 hours, the reaction mixture was precipitated from brine, and centrifuged. The sediment was collected and dried. It gave a purple product. ¹H NMR (300 MHz, d₆-DMSO): δ=11.02 (1H, br, COOH), 8.57 (1H, m, Ar—H), 8.50 (1H, m, Ar—H), 8.19 (1H, m, NHC═O), 7.93 (3H, m, NH+Ar—H), 7.73 (1H, m, Ar—H), 7.70 (1H, m, Ar—H), 7.64 (2H, m, NH₂), 6.92 (1H, d, Ar—H), 6.86 (1H, s, triazole-H), 6.62 (2H, m, Ar—H), 4.60 (2H, m, NCH₂C═N), 4.49 (2H, m, OCH₂CN), 4.34 (1H, m, NCHC═O), 4.24 (1H, m, CH₂), 4.11 (1H, m, CH₂), 3.56 (17H, br, OCH₂), 2.21 (2H, m, CH₂), 1.92 (4H, m, CH₂CH₂CH₂), 1.21 (2H, m, CH₂).

### Example 11D

### Hydrophilic Folate-Fluorescein

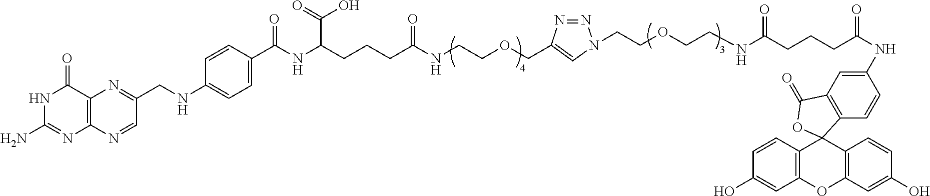

Hydrophilic folate alkyne (0.020 g, 30.5E-6 mol) and hydrophilic fluorescein azide (20.5 mg, 30.5E-6 mol) were dissolved in DMSO, followed by addition of CuSO₄.5H₂O (8 mg, 32.0E-6 mol), sodium ascorbate (12 mg, 62.5E-6 mol) and water (0.05 mL). The mixture turned deep brown and was stirred at room temperature. After 48 h, the reaction mixture was precipitated from brine, and centrifuged. The sediment was collected and dried. It gave 24.4 mg (60%) black product. ¹H NMR (300 MHz, d₆-DMSO): δ=10.34 (1H, br, COOH), 10.08 (2H, br, OH), 8.60 (1H, m, Ar—H), 8.30 (1H, s, NH), 8.19 (1H, m, NH), 8.02 (1H, m, triazole-H), 7.87 (2H, t, Ar—H+ NH), 7.78 (1H, m, Ar—H), 7.64 (2H, m, NH₂), 7.15 (1H, s, Ar—H), 6.90 (2H, m, Ar—H), 6.64 (2H, m, Ar—H), 6.55 (4H, m, Ar—H), 4.48 (4H, m, CH₂N), 4.02 (3H, m, NH+CH₂), 3.78 (2H, m, OCH₂CH₂N), 3.45 (12H, br, OCH₂), 3.31 (10H, m, OCH₂CH₂O), 3.17 (5H, m, CH₂C), 2.52 (2H, m, NC═OCH₂), 2.35 (2H, m, NC═OCH₂), 2.14 (2H, m, CH₂CH₂CH₂), 1.97 (4H, m, CH₂CH₂CH₂), 1.82 (2H, t, CH₂), 1.15 (2H, m, CH₂).

### Example 12

### Labeling Cells with Markers

Various cells were labeled with markers according to certain embodiments of the present invention, and luminescence was observed from the markers.

### Example 12A

### Labeling Cells with Folate Receptor by Marker Comprising Folate Binding Agent

Two groups of cells were grown. First, control cells of the strain NIH-3T3 were grown in medium that contained folic acid. Those cells were chosen because they are known to have few folate receptors on their surfaces. Moreover, the folic acid in the medium is believed to provide plenty of folic acid to the cells so that the cells do not overexpress the genes for producing folate receptors. The experimental cells, KB cells, were grown in medium without folic acid. Starving KB cells known for surface folate receptors causes overexpression of the genes that produce folate receptors, resulting in an abundance of folate receptors on the KB cells.

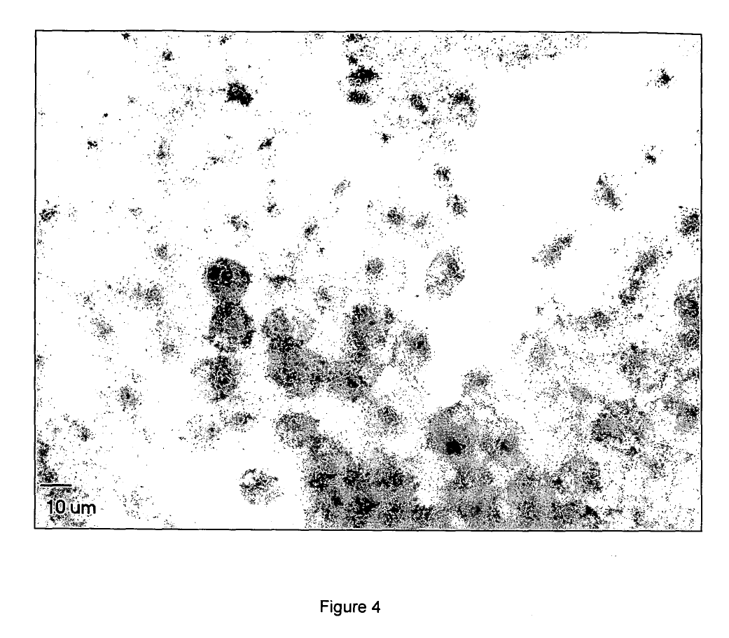

To otherwise identical samples of cells, a nanoparticle/PEG/(folate and hydrophilic fluorescein) marker made as described in Example 10F were introduced, incubated for 24 hours, and unbound markers were rinsed away. A confocal microscope equipped to image photoluminescence was set to parameters that were maintained for both the experimental and control measurements. It was observed that the control NIH-3T3 cells exhibited low non-specific binding of the marker comprising a nanoparticle, folate and hydrophilic fluorescein, but the experimental KB cells showed high specific binding affinity for the same marker. A color-inverted composite image of the photoluminescence under channel pass filters (DAPI, FITC and Cy3) from the labeled KB cells is shown in FIG. 4. To obtain those images, a mercury arc lamp output passed through the filters individually, and the emission was observed for ranges of wavelengths for each excitation filter. That is, three images were taken, one through each of the three channel pass filters, and then the three images were combined to form FIG. 4. The excitation and observation wavelength ranges were:

| Filter | λₑₓ (nm) | λ_{observe} (nm) |
|---|---|---|
| DAPI | 377 ± 50 | 447 ± 50 |
| FITC | 472 ± 30 | 520 ± 35 |
| Cy3 | 531 ± 40 | 593 ± 40 |

### Example 12B

### Employing a Streptavidin Bridge to Bind a Marker to a Cell

J774.A1 murine macrophage cells were grown and exposed to biotinylated anti-CD 16/32 primary antibody. Next, the bound antibody was exposed to streptavidin. Markers comprising nanoparticles/PEG passivation agent/fluorescein isothiocyanate (“FITC”) chromophore and biotin binding agent were added to target the streptavidin bound to the biotinylated antibody anchored to the cells. The markers were found to have 30 FITC per nanoparticle (as determined by UV-Vis spectroscopy) and 14 biotin per nanoparticle (as determined by HABA assay). Photoluminescence revealed good specific binding of the markers to the cells.

### INDUSTRIAL APPLICABILITY

The markers and kits of the present invention can be employed in many in vitro diagnostic settings. In some embodiments, those markers and kits are adapted for anatomic, physiologic, biochemical (immunologic), or molecular (genetic) parameters that are associated with the presence and severity of a specific disease or disorder. For example, some embodiments provide markers and kits for immunoassays, while other embodiments provide markers and kits for molecular assays, while still other embodiments provide markers and kits for histology or cytology. Some embodiments provide markers and kits for one or more of high-throughput screening micro-arrays, ELISA assays, western blots, flow cytometry, cell imaging, tumor margin assessment, pathogen detection, genetic disease detection, and other biological probes. Other embodiments provide markers and kits for one or more of nucleic acid biomarker detection, immunohistochemistry, multiplex labeling, and fluorescence resonance energy transfer (FRET). Markers and kits, in some embodiments, are adapted for one or more of blood sugar testing, illegal drug use testing, pregnancy testing, paternity testing, blood-type testing, and infectious disease testing.

Still other embodiments provide markers and kits for one or more of crime scene investigations, fire and arson investigation, and security screening for explosives, firearms, and illegal drugs. Additional embodiments provide markers and kits for industrial process monitoring.

Still other embodiments provide markers and kits for agricultural testing, such as, for example, so a manufacturer of proprietary seed can test whether certain crops have been grown with proprietary seed. Still other embodiments for agricultural use provide markers and kits for detecting one or more of infectious disease, ripeness based on biological markers thereof, and the presence of pesticides, herbicides, and pollutants (e.g., whether a given produce is “organically grown”). Still other embodiments provide markers and kits for veterinary use, detecting one or more of infectious disease, medical disorder, pregnancy, other physiological status such as “heat,” and genetic heritage including susceptibility to illness.

It will be appreciated that the foregoing examples, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention. Further, it is recognized that many embodiments may be conceived that do not achieve all of the advantages of some embodiments, yet the absence of a particular advantage shall not be construed to necessarily mean that such an embodiment is outside the scope of the present invention.

What is claimed is:

1. A marker, comprising:
at least one binding agent; and
at least one chromophore.

2. The marker of claim 1, further comprising
at least one nanoparticle comprising at least one carbon core and at least one passivation agent coupled to the at least one carbon core,
wherein the at least one carbon core is less than about 100 nm in size, and
wherein the at least one nanoparticle is luminescent.

3. The marker of claim 2, wherein the at least one passivation agent is chosen from O,O′-bis(3-aminopropyl)poly(ethylene glycol), polyoxyalkyleneamine, and poly(propionylethylenimine-co-ethylenimine).

4. The marker of claim 2, wherein the at least one binding agent is chosen from biotin, folic acid, streptavidin, and combinations thereof.

5. The marker of claim 2, wherein the at least one chromophore is chosen from fluorescein, fluorescein isothiocyanate, coumarin, rhodamine, pyrene, anthracene, and combinations thereof.

6. The marker of claim 2, further comprising at least one linker.

7. The marker of claim 6, wherein the at least one linker is chosen from alkyne linker, azide linker, 1,11-diazido-3,6,9-trioxaundecane, 1-amino-11-azido-3,6,9-trioxaundecane, and combinations thereof.

8. The marker of claim 1, having the structure:

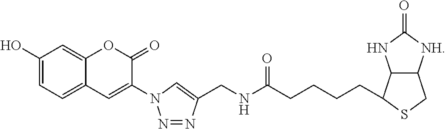

9. The marker of claim 1, having the structure:

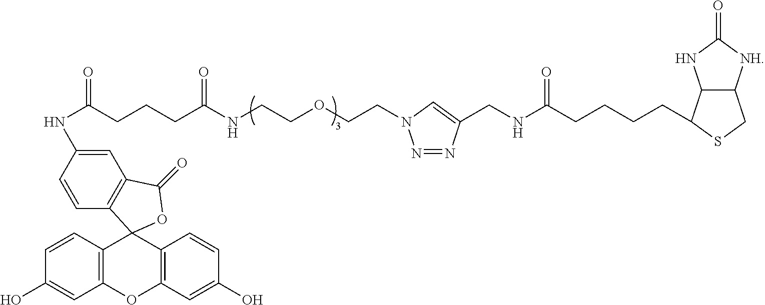

10. The marker of claim 1, having the structure:

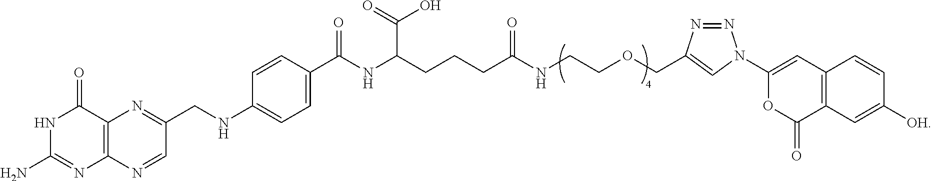

11. The marker of claim 1, having the structure:

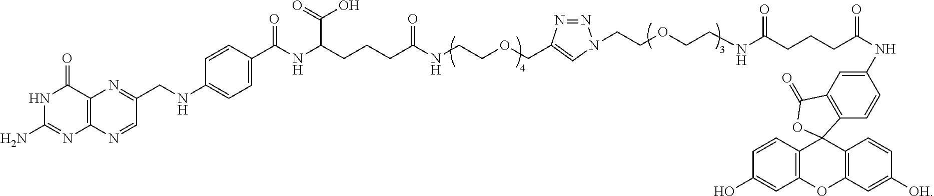

12. A method of investigating at least one analyte in vitro, comprising:
correlating the at least one analyte with at least one marker; and
observing the luminescent emission of the at least one marker;
wherein the luminescent emission is chosen from chemiluminescence, electroluminescence, thermal luminescence, sonoluminescence, and combinations thereof.

13. The method of claim 12, wherein the at least one marker comprises:
at least one nanoparticle comprising at least one carbon core and at least one passivation agent coupled to the at least one carbon core, wherein the at least one carbon core is less than about 100 nm in size.

14. The method of claim 12, wherein the at least one marker comprises one or more binding agents chosen from antigens, antibodies, hormones, DNA fragments, polysaccharides, proteins, peptides, cell-surface receptors, fractions of any of the foregoing, or a combination of two or more of any of the foregoing.

15. A method of investigating at least one analyte in vitro, comprising:
correlating the at least one analyte with at least one marker, wherein the correlating comprises forming at least one sandwich complex; and
observing the luminescent emission of the at least one marker;
wherein the at least one marker comprises at least one nanoparticle comprising at least one carbon core and at least one passivation agent coupled to the at least one carbon core, and wherein the at least one carbon core is less than about 100 nm in size.

16. The method of claim 15, wherein the sandwich complex comprises:
at least one immobilized antibody;
the at least one analyte, which comprises at least one antigen, bound to the at least one immobilized antibody; and
the at least one marker, which comprises at least one additional antibody, bound to the at least one antigen.

17. The method of claim 15, wherein the sandwich complex comprises:
at least one immobilized antigen;
the at least one analyte, which comprises at least one primary antibody, bound to the at least one immobilized antigen; and
the at least one marker, which comprises at least one secondary antibody, bound to the at least one primary antibody.

18. A kit for in vitro diagnosis, comprising:
at least one marker that is adaptable to correlate with at least one analyte;
wherein the at least one marker comprises at least one nanoparticle comprising at least one carbon core and at least one passivation agent coupled to the at least one carbon core, wherein the at least one carbon core is less than about 100 nm in size, and wherein the at least one nanoparticle is luminescent.

19. The kit of claim 18, wherein the at least one marker is immobilized on at least one structure.

20. The kit of claim 18, wherein the at least one marker comprises at least one molecular beacon.

21. The kit of claim 18, wherein the structure comprises a DNA binding array.

22. The kit of claim 18, wherein the structure comprises a RNA binding array.

23. A kit for in vitro diagnosis, comprising:
at least one marker that is adaptable to correlate with at least one analyte;
wherein the at least one marker comprises at least one nanoparticle comprising a carbon core and at least one passivation agent coupled to the carbon core, wherein the carbon core is less than about 100 nm in size; and
wherein the at least one marker exhibits increased luminescence either in the presence or absence of the analyte.

24. The kit of claim 23, wherein the at least one marker comprises at least one quencher.

25. The kit of claim 24, wherein the at least one marker comprises at least one molecular beacon to which the at least one nanoparticle and the at least one quencher are bound.
